(19) United States
(12) Patent Application Publication
(10) Pub. No.: US 2011/0097312 A1
(43) Pub. Date: Apr. 28, 2011

(54) ANTI-CANCER VACCINES

(75) Inventor: Jeffrey Molldrem, Houston, TX (US)

(73) Assignee: BOARD OF REGENTS, THE UNIVERSITY OF TEXAS SYSTEM

(21) Appl. No.: 12/867,083

(22) PCT Filed: Feb. 13, 2009
(86) PCT No.: PCT/US09/33987

§ 371 Date: Dec. 14, 2010

Related U.S. Application Data
(60) Provisional application No. 61/029,141, filed on Feb. 15, 2008.

(51) Int. Cl. A61K 39/00; A61K 35/12

(52) U.S. Cl. 424/93.71; 424/277.1

(57) ABSTRACT

The present provides tumor-associated HLA-restricted antigens, and in particular HLA-A2 restricted antigens, as immunogenic compositions for treating and/or preventing breast cancer in an individual. In specific aspects, PR1 peptide or a derivative thereof, or a myeloperoxidase peptide, or a cyclin E1 or E2 peptide is provided in methods and compositions for breast cancer treatment and/or prevention. Such peptides can be used to elicit specific CTLs that preferentially attack breast cancer based on overexpression of the target protein cells.

This application claims priority to U.S. Provisional Patent Application Ser. No. 61/029,141, filed Feb. 15, 2008, which is incorporated by reference herein in its entirety.

The government owns rights in the present invention pursuant to grant number CA81247 from the National Cancer Institute of the National Institutes of Health.

### FIELD OF THE INVENTION

The present invention relates generally to the fields of cancer and immunotherapy. More particularly, it concerns the identification of immunotherapeutic peptides and the development of peptide vaccines for the treatment and prevention of cancer, including breast cancer.

### BACKGROUND OF THE INVENTION

The immune system has long been implicated in the control of cancer and is an attractive target for cancer therapy and prevention. In particular, it would be useful to employ the body's own immune system to directly and specifically target cancer cells while leaving normal cells unharmed. In melanoma and breast cancer, for example, many peptide antigens have been identified as targets of tumor-specific CTL. What is clear from these studies is that nearly all of the tumor antigens identified are derived from normal tissue proteins (Nanda and Sercarz, 1995; Boon et al., 1997). It is now accepted that many self-antigenic determinants have not induced self-tolerance and that these peptide determinants supply target structures for autoimmune attack (Nanda and Sercarz, 1995; Rosenberg and White, 1996; Goulmy et al., 1996; Dermime et al., 1995). Since these proteins are often aberrantly expressed or overexpressed in the tumor, there is relative tumor specificity by CTL that recognize these epitopes (Pardoll, 2002; Pardoll, 1994; Bocchia et al., 1996; Pinilla-Ibarz et al., 2000). Similarly in leukemia, CTL immunity to the Wilm's tumor antigen WT-1, which is aberrantly expressed in various forms of leukemia, has been demonstrated to kill CML CD34+ progenitor cells (Gao et al., 2000).

Melanoma peptide antigens that are derived from MAGE-3 proteins, for example, are presented to melanoma-specific CTLs by HLA-A1 and HLA-A2 (Nanda and Sercarz, 1995; Boon et al., 1997; Rosenberg and White, 1996). This protein belongs to a family of proteins which are expressed in melanoma cells and in normal testis. A MAGE-3 derived peptide was determined to be immunogenic by separate groups using different techniques, one using an immunological method (Pardoll, 2002) and the other a genetic method that uses tumor antigen-deficient mutants (Nanda and Sercarz, 1995). Recently, a phase I clinical trial using MAGE-3 to vaccinate melanoma patients resulted in some clinical responses (Pardoll, 1994). In addition, tyrosinase, gp100, and Melan-A-MART-1 are also normal self-proteins specific to the melanocyte lineage and T-cells specific for determinants on each of these antigens can be found in a large majority of melanoma patients (Sturrock et al., 1992; Chen et al., 1994). Two recent phase II vaccine trials demonstrated clinical efficacy of active immunotherapy using these target antigens as a peptide vaccine or as a antigen-pulsed dendritic cell vaccine.

PR1, an HLAA2.1-restricted nonamer derived from proteinase 3 (P3), was identified as a leukemia-associated antigen (Molldrem et al., 2000; Molldrem et al., 1996; Molldrem et al., 1997; Molldrem et al., 1999; Molldrem et al., 2003 each incorporated herein by reference in their entirety). The finding that PR1 is a leukemia-associated antigen has been independently confirmed by Burchert et al. (2002) and Scheibenbogen et al. (2002). These studies have thus established PR1 as a human leukemia-associated antigen and have established that PR1-specific CTL contribute to the elimination of CML (see US 2006/0045883, for example). However, there is absence of evidence showing PR1 efficacy outside of leukemia.

Although some tumor specific antigens have been identified as putative immunotherapeutic targets, there still is a great need in the art to identify more antigens and develop immunotherapeutic methods that target breast cancers, for example. New approaches for treatment of cancers are therefore needed.

### SUMMARY OF THE INVENTION

The present invention concerns methods and compositions concerning an immunogenic composition, including a vaccine, for example, comprising a first tumor associated HLA restricted peptide and methods employing the composition. The HLA-restricted peptide may be an HLA-A2 restricted peptide, such as proteinase-3 peptide of VLQELNVTV (SEQ ID NO:1; “PR1”), or a modified PR1-derived peptide selected from the group consisting of VLQELWTV (SEQ ID NO:2), VLQELNVKV (SEQ ID NO:3), VLQELWKV (SEQ ID NO:4) and VMQELWTV (SEQ ID NO:5), or a fragment thereof. In other embodiments, peptides for myeloperoxidase or cyclin E1 are similarly employed.

In particular cases, the immunogenic composition may further comprise an adjuvant, such as complete Freund's adjuvant, incomplete Freund's adjuvant, alum, Bacillus Calmette-Guerin, agonists and modifiers of adhesion molecules, tetanus toxoid, imiquinod, montanide, MPL, and QS21, for example. The immunogenic composition may also further comprise an immunostimulant.

The immunogenic composition may comprise more than one peptide, and the multiple peptides may depend on the breast tumor to be treated, and/or the HLA type of the individual. The immunogenic composition may further comprise an antigen presenting cell, such as a dendritic cell, and more particularly a dendritic cell pulsed or loaded with the peptide and used as a cellular vaccine to stimulate T cell immunity against the peptide, and thereby against the tumor.

The immunogenic composition may further comprise a second tumor-associated HLA-restricted peptide. The immunogenic composition may further comprise a third, fourth or fifth tumor-associated HLA-restricted peptide. The second, third, fourth or fifth tumor-associated HLA-restricted peptide may be an HLA-A2, HLA-A3, HLA-A11, HLA-B7, HLA-B27 or HLA-B35 restricted peptide, for example.

In another embodiment, there is provided a method for treating and/or preventing breast cancer in an individual comprising administering to the individual a therapeutically effective amount of an immunogenic composition of PR1 or a PR1 derivative thereof, a PR3 peptide, a myeloperoxidase or cyclin E1 or E2 peptide, or a mixture thereof. The immunogenic composition may be administered more than once. The therapeutically effective amount may be in the range of 0.20 mg to 5.0 mg, or in the range of 0.025 mg to 1.0 mg, or in the range of 2.0 mg to 5.0 mg of the peptide, for example.

In specific embodiments of the invention, an adjuvant and a PR1 peptide, a PR1 derivative thereof, a PR3 peptide, a myeloperoxidase, a cyclin E1 peptide, a cyclin E2 peptide, or a mixture thereof are injected subcutaneously into an individual, followed by injection at the same site of an immunomodulatory agent having the ability to boost an immune response to an immunogenic composition, such as a vaccine, in specific embodiments.

In certain cases, the individual that is provided methods or compositions of the invention is an individual known to have breast cancer, suspected of having breast cancer, or is at risk for having breast cancer. The cancer may be recurrent cancer. The cancer may be metastatic, in certain cases. An individual at risk for having breast cancer is an individual that has already had breast cancer, has a family history of breast cancer, has a history of benign breast tumors, is a smoker, is older than 45 years old, or has mutations in BRCA1, BRCA2, p53, ATM, CHEK2, or PTN, for example.

In particular cases, the breast cancer to be treated is present in a woman or a man. The breast cancer may be any type of breast cancer, but in specific embodiments the breast cancer is estrogen receptor (ER) positive, ER negative, progesterone receptor (PR) positive, PR negative, HER-2 positive, HER-2 negative, inflammatory breast cancer, non-inflammatory breast cancer, noninvasive breast cancer (including ductal carcinoma in situ, for example), infiltrating breast cancer (including invasive ductal carconima and invasive lobular carcinoma, for example), medullary carcinoma, mucinous (colloid) carcinoma; Paget's disease of the breast, tubular carcinoma, Phylloides tumor, metaplastic carcinoma, sarcoma, micropapillary carcinoma, adenoid cystic carcinoma, and so forth. The breast cancer may arise in a duct, a lobule, fibrous connective tissue, blood vessels, or lymphatic system in the breast. The breast cancer may be Grade 1, Grade 2, or Grade 3.

In specific embodiments, the method may use the immunogenic composition administered subcutaneously, systemically, intravenously, intra-arterially, intra-peritoneally, intramuscularly, intradermally, intratumorally, orally, dermally, nasally, buccally, rectally, vaginally, by inhalation, or by topical administration, for example. The immunogenic composition may be administered locally, by direct intratumoral injection, by injection into tumor vasculature or by an antigen-presenting cell pulsed or loaded with the peptide, wherein the antigen presenting cell may be a dendritic cell. The antigen-presenting cell may comprise one or more distinct peptides. The method may utilize a cellular vaccine.

The method may further comprise treating an individual with a second anticancer agent, wherein the second anticancer agent is a therapeutic polypeptide, peptide (including one or more peptides of the invention), a nucleic acid encoding a therapeutic polypeptide, a chemotherapeutic agent, a biological and/or small molecule targeting agent, an immunomodulatory agent, a radiotherapeutic agent, or a combination thereof, for example. The second anticancer agent may be administered simultaneously with the vaccine, or administered at a different time than the immunogenic composition. The second anticancer agent may be Herceptin®, in certain cases. The immunomodulatory agent may be GM-CSF, CD40 ligand, anti-CD28 mAbs, anti-CTL-4 mAbs, anti-4-1BB (CD137) mAbs, and/or an oligonucleotide. The chemotherapeutic agent may be doxorubicin, daunorubicin, dactinomycin, mitoxantrone, cisplatin, procarbazine, mitomycin, carboplatin, bleomycin, etoposide, teniposide, mechlroethamine, cyclophosphamide, ifosfamide, melphalan, chlorambucil, ifosfamide, melphalan, hexamethylmelamine, thiopeta, busulfan, carmustine, lomustine, semustine, streptozocin, dacarbazine, adriamycin, 5-fluorouracil (5FU), camptothecin, actinomycin-D, hydrogen peroxide, nitrosurea, plicomycin, tamoxifen, taxol, transplatinum, vincristin, vinblastin, a TRAIL R1 and R2 receptor antibody or agonist, dolastatin-10, bryostatin, annamycin, mylotarg, sodium phenylacetate, sodium butyrate, methotrexate, dacitabine, imatinab mesylate (Gleevec), interferon-α, bevacizumab, cetuximab, thalidomide, bortezomib, gefitinib, erlotinib, azacytidine, 5-AZA-2′ deoxycytidine, Revlimid, 2C4, an anti-angiogenic factor, a signal transducer-targeting agent, interferon-γ, IL-2 and IL-12.

A biological and/or small molecule targeting agent may be further defined as a monoclonal antibody or a small molecule targeted to tyrosine kinases, for example. In specific embodiments, the biological and/or small molecule targeting agent is an anti-angiogenesis agent (for example, Avastin®), a tyrosine kinase inhibitor (for example, Sutent® or Nexavar®), or an anti-epidermal growth factor (EGF) agent. The biological and/or small molecule targeting agent may target Her-2, for example. In specific cases, the biological and/or small molecule targeting agent targets EGF or its receptor. An exemplary biological and/or small molecule targeting agent includes Herceptin®.

In yet another embodiment, there is provided a method for treating or preventing cancer in an individual comprising (a) contacting CTLs of the patient with a PR1 peptide, a PR1-derived peptide, a myeloperoxidase peptide, a cyclin E1 or cyclin E2 peptide, or a mixture thereof; and (b) administering a therapeutically effective amount of the CTLs of step (a) to the individual. The method may further comprise expanding the CTL's by ex vivo or in vivo methods prior to administration. Contacting may comprise providing an antigen presenting cell loaded with the peptide or that expresses the peptide from an expression construct. The method may further comprise providing CTLs transfected with a T cell receptor specific for the peptide. The therapeutically effective amount of CTL cells required to provide therapeutic benefit may be from about 0.1×10⁵ to about 5×10⁷ cells per kilogram weight of the subject, for example.

In still yet another embodiment, there is provided a method for treating and/or preventing a cancer in an individual comprising administering to the patient a therapeutically effective amount of an immunogenic composition comprising an expression construct encoding a PR1 peptide. The expression construct may be a non-viral expression construct or a viral expression construct. The expression construct may also encode a second tumor associated peptide.

As used herein the specification, “a” or “an” may mean one or more. As used herein in the claim(s), when used in conjunction with the word “comprising”, the words “a” or “an” may mean one or more than one. As used herein “another” may mean at least a second or more. Some embodiments of the invention may consist of or consist essentially of one or more elements, method steps, and/or methods of the invention. It is contemplated that any method or composition described herein can be implemented with respect to any other method or composition described herein.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows aberrant expression of PRTN3 and ELA2 in breast cancer.

FIG. 2 demonstrates that PR1-CTL specifically lyse HLA-A2⁺ breast cancer cells.

### DESCRIPTION OF ILLUSTRATIVE EMBODIMENTS

This application incorporates U.S. patent application Ser. No. 10/926,852, filed Aug. 26, 2004, by reference herein in its entirety.

### I. THE PRESENT INVENTION

The present invention serves to overcome the deficiencies in the art by providing HLA-restricted peptides, derived from myeloid self-proteins, that can be used to elicit peptide-reactive CTL that preferentially target breast cancer. These peptides and the peptide-reactive CTL will be used in immunogenic compositions, such as vaccines or as adoptive cellular immunotherapy, to treat patients with breast cancer.

The findings of the present invention indicate that the exemplary PR1 peptide is an important tumor antigen for CTL immune responses against breast cancer, and provide direct evidence that an antigen-specific T cell response contributes to its control. In specific embodiments of the invention, normal healthy donors have existent CTL immunity to PR1 and that breast cancer patients who have a cytogenetic remission after treatment also have effective PR1-specific CTL immunity toward their breast cancer cells, while patients without cytogenetic responses do not; thus, PR1 acts as a breast cancer-associated tumor antigen, in particular aspects of the invention. In a specific embodiment, vaccination with PR1 peptide enhances immunity toward breast cancer in a fashion similar to that observed with leukemias.

Although some embodiments described herein reflect disclosure for PR1, such embodiments may be similarly applied to PR1-derived peptides, myeloperoxidase peptides, and cyclin E1 or cyclin E2 peptides.

### II. DEFINITIONS

The phrases “isolated” or “biologically pure” refer to material which is substantially or essentially free from components which normally accompany the material as it is found in its native state. Thus, isolated peptides in accordance with the invention preferably do not contain materials normally associated with the peptides in their in situ environment.

“Major histocompatibility complex” or “MHC” is a cluster of genes that plays a role in control of the cellular interactions responsible for physiologic immune responses. In humans, the MHC complex is also known as the HLA complex. For a detailed description of the MHC and HLA complexes (see Paul, 1993).

“Human leukocyte antigen” or “HLA” is a human class I or class II major histocompatibility complex (MHC) protein (see, e.g., Stites, 1994).

An “HLA supertype or family”, as used herein, describes sets of HLA molecules grouped on the basis of shared peptide-binding specificities. HLA class I molecules that share somewhat similar binding affinity for peptides bearing certain amino acid motifs are grouped into HLA supertypes. The terms HLA superfamily, HLA supertype family, HLA family, and HLA xx-like supertype molecules (where xx denotes a particular HLA type), are synonyms.

An “immunogenic composition” as used herein refers to a composition that elicits an immune response in an individual. The composition may or may not be a vaccine. In specific aspects, the peptides of the invention support an immune reaction against breast cancer.

The term “motif” refers to the pattern of residues in a peptide of defined length, usually a peptide of from about 8 to about 13 amino acids for a class I HLA motif and from about 6 to about 25 amino acids for a class II HLA motif, which is recognized by a particular HLA molecule. Peptide motifs are typically different for each protein encoded by each human HLA allele and differ in the pattern of the primary and secondary anchor residues.

A “supermotif” is a peptide binding specificity shared by HLA molecules encoded by two or more HLA alleles. Thus, a preferably is recognized with high or intermediate affinity (as defined herein) by two or more HLA antigens.

“Cross-reactive binding” indicates that a peptide is bound by more than one HLA molecule; a synonym is degenerate binding.

A “protective immune response” refers to a CTL and/or an HTL response to an antigen derived from an infectious agent or a tumor antigen, which prevents or at least partially arrests disease symptoms or progression. The immune response may also include an antibody response which has been facilitated by the stimulation of helper T cells.

### III. HLA-RESTRICTED PEPTIDES

The present provides an immunogenic composition comprising a tumor associated HLA restricted peptide. “Human leukocyte antigen” or “HLA” is a human class I or class II major histocompatibility complex (MHC) protein (see, e.g., Stites, 1994). An “HLA supertype or family,” as used herein, describes sets of HLA molecules grouped on the basis of shared peptide-binding specificities. HLA class I molecules that share somewhat similar binding affinity for peptides bearing certain amino acid motifs are grouped into HLA supertypes. The terms HLA superfamily, HLA supertype family, HLA family, and HLA xx-like supertype molecules (where xx denotes a particular HLA type), are synonyms. HLA-restricted molecules of the present invention may include any HLA types, but in specific embodiments they include HLA-A2, HLA-A3, HLA-A11, HLA-B7, HLA-A24, HLA-B27, or HLA-B35; but are not limited to such.

Exemplary HLA-restricted antigens/peptides include, but are not limited to the following: 707 alanine proline (707-AP); alpha (α)-fetoprotein (AFP); adenocarcinoma antigen recognized by T cells 4 (ART-4); B antigen (BAGE); β-catenin/m, β-catenin/mutated; breakpoint cluster region-Abelson (Bcr-abl); CTL-recognized antigen on melanoma (CAMEL); carcinoembryonic antigen peptide-1 (CAP-1); caspase-8 (CASP-8); cell-division-cycle 27 mutated (CDC27m); cycline-dependent kinase 4 mutated (CDK4/m); carcinoembryonic antigen (CEA); cancer/testis (antigen) (CT); cyclophilin B (Cyp-B); differentiation antigen melanoma (the epitopes of DAM-6 and DAM-10 are equivalent, but the gene sequences are different. DAM-6 is also called MAGE-B2 and DAM-10 is also called MAGE-B1) (DAM); elongation factor 2 mutated (ELF2M); Ets variant gene 6/acute myeloid leukemia 1 gene ETS (ETV6-AM1); glycoprotein 250 (G250); G antigen (GAGE); N-acetylglucosaminyltransferase V (GnT-V); glycoprotein 100 Kd (Gp100); helicose antigen (HAGE); human epidermal receptor-2/neurological (HER-2/neu); arginine (R) to isoleucine (I) exchange at residue 170 of the α-helix of the α2-domain in the HLA-A2 gene (HLA-A*0201-R1701); human papilloma virus E7 (HPV-E7); heat shock protein 70-2 mutated (HSP70-2M); human signet ring tumor-2 (HST-2); human telomerase reverse transcriptase (hTERT or hTRT); intestinal carboxyl esterase (iCE); name of the gene as it appears in databases (KIAA0205); L antigen (LAGE); low density lipid receptor/GDP-L-fucose (LDLR/FUT); β-D-galactosidase 2-α-L-fucosyltransferase; melanoma antigen (MAGE); melanoma antigen recognized by T cells-1/Melanoma antigen A (MART-1/Melan-A); melanocortin 1 receptor (MC1R); myosin mutated (Myosin/m); mucin 1 (MUC1); melanoma (MUM-1, -2, -3); ubiquitous mutated 1, 2, 3; NA cDNA clone of patient M88 (NA88-A); New York-esophageous 1 (NY-ESO-1); protein 15 (P15); protein of 190 (p190 minor bcr-abl); KD bcr-abl; promyelocytic leukaemia/retinoic acid receptor α (Pml/RARα); preferentially expressed antigen of melanoma (PRAME); prostate-specific antigen (PSA); prostate-specific membrane antigen (PSM); renal antigen (RAGE); renal ubiquitous 1 or 2 (RU1 or RU2); sarcoma antigen (SAGE); squamous antigen rejecting tumor 1 or 3 (SART-1 or SART-3); translocation Ets-family leukemia/acute myeloid leukemia 1 (TEL/AML1); triosephosphate isomerase mutated (TPI/m); tyrosinase related protein 1, or gp75 (TRP-1); tyrosinase related protein 2 (TRP-2); TRP-2/intron 2 (TRP-2/INT2); Wilms' tumor gene (WT1) or any such HLA-restricted antigen or peptide known to one of ordinary in the art.

In particular embodiments, the present invention contemplates the use of HLA-restricted peptide for treating cancers.

### IV. PR1 PEPTIDE AND IMMUNOGENIC COMPOSITIONS

It has been shown that a small peptide called PR1, a portion of the larger molecule of proteinase 3 (P3) found in myeloid leukemia cells, can be used to generate immune cells, particularly, cytotoxic T lymphocytes (CTL). PR1 is a 9 aa peptide comprising amino acid 169-177, that binds to HLA-A2.1, thereby eliciting CTL from an HLA-A2.1+ normal donor in vitro. These PR1-specific CTL show preferential cytotoxicity toward allogeneic HLA-A2.1+ myeloid leukemia cells over HLA-identical normal donor marrow (Molldrem et al., 1996). PR1-specific CTL also inhibit colony-forming unit granulocyte-macrophage (CFU-GM) from the marrow of CML patients, but not CFU-GM from normal HLA-matched donors (Molldrem et al., 1997). These CTL, generated from normal healthy donors, preferentially kill leukemia cells while leaving normal bone marrow cells unharmed. More recently, it was found that CML patients who enter remission after treatment with either bone marrow transplant (BMT) or interferon have highly increased numbers of very effective PR1-specific CTL that kill their leukemia cells. PR1 is therefore the first peptide antigen identified that can elicit specific CTL lysis of fresh human myeloid leukemia cells. In particular aspects of the invention, PR1 is able to elicit a similar reaction for breast cancer cells.

The leukemia-associated antigen PR1 is derived from both proteinase 3 and neutrophil elastase (NE) proteins, in particular aspects of the invention. Furthermore, in addition to P3, PR1 is also processed and presented by NE, which results in enhanced immunogenicity of the peptide compared to peptides derived from a single protein, in specific embodiments of the invention. Redundancy of proteins may also lessen the impact of tumor-loss variants after PR1-based immunotherapy, at least in certain cases.

### V. PROTEINASE-3 (PR3) IMMUNOGENIC COMPOSITIONS

Pr3 is a 26 kDa neutral serine protease that is stored in primary azurophil granules and is maximally expressed at the promyelocyte stage of myeloid differentiation (Sturrock et al., 1992; Chen et al., 1994; Muller-Berat et al., 1994; Lewin et al., 2002; Behre et al., 1999). The human gene contains 5 exons, is localized on chromosome 19p and has been cloned (Sturrock et al., 1992). Pr3 is overexpressed in a variety of myeloid leukemia cells, for example, including 75% of CML patients, approximately 50% of acute myeloid leukemia patients, and approximately 30% of the cases of myelodysplastic syndrome patients (Dengler et al., 1995).

In specific embodiments, Pr3 is utilized as a target for vaccine and T cell-directed therapy. In additional embodiments, there is the use of a synthetic peptide derived from Pr3 as the immunizing antigen in a breast cancer vaccine, for example.

### VI. MYELOPEROXIDASE (MPO), CYCLIN E1, AND CYCLIN E2 PEPTIDES AND IMMUNOGENIC COMPOSITIONS

The invention also provides peptides of myeloperoxidase (MPO), another myeloid-restricted protein, which is a heme protein synthesized during early myeloid differentiation that constitutes the major component of neutrophil azurophilic granules. Produced as a single chain precursor, myeloperoxidase is subsequently cleaved into a light and heavy chain. The mature myeloperoxidase is a tetramer composed of 2 light chains and 2 heavy chains (Franssen et al., 1996). This enzyme produces hypohalous acids central to the microbicidal activity of netrophils. Importantly, MPO (like Pr3) is overexpressed in a variety of myeloid leukemia cells including 75% of CML patients, approximately 50% of acute myeloid leukemia patients, and approximately 30% of the cases of myelodysplastic syndrome patients (Williams et al., 1994). While Pr3 is the target of autoimmune attack in Wegener's granulomatosis, MPO is the target antigen in small vessel vasculitis (Franssen et al., 1996; Brouwer et al., 1994; Molldrem et al., 1996) respectively, with evidence for both T-cell and antibody immunity in patients with these diseases. Wegener's granulomatosis is associated with production of cytoplasmic antineutrophil cytoplasmic antibodies (cANCA) with specificity for Pr3 (Molldrem et al., 1997), while microscopic polyangiitis and Churg-Strauss syndrome are associated with perinuclear ANCA (pANCA) with specificity for MPO (Molldrem et al., 1999; Savage et al., 1999). T-cells taken from such leukemia patients proliferate in response to crude extracts from neutrophil granules and to the purified proteins (Brouwer et al., 1994; Yee et al., 1999).

In addition, cyclin E is pertinent to embodiments of the invention. Cyclin E is constitutively expressed in some tumor cells independent of the cell cycle, and aberrantly expressing cyclin E contributes to tumorigenesis as a result of chromosomal instability. Both cyclin E1 and cyclin (a homologue of cyclin E1) have restricted tissue distribution. Furthermore, cyclin E1 and E2 are over-expressed in hematological malignancy and cyclin E1 and E2 peptide-specific CTLs can recognize both peptides with HLA-A2. The invention further provides peptides of cyclin E1 and cyclin E2 in the context of breast cancer treatment and/or prevention.

### VII. TUMOR-ASSOCIATED HLA-RESTRICTED PEPTIDES AND IMMUNOGENIC COMPOSITIONS

In certain embodiments, the present invention concerns tumor-associated HLA-restricted peptide or antigen compositions comprising at least one HLA-restricted peptide, such as proteinase3 (P3 or Pr3) or myeloperoxidase (MYO) or Cyclin E1 for use as an immunogenic composition in treating breast cancers.

Iit is contemplated that administration of the immunogenic composition with a tumor-associated HLA-restricted peptides, or polypeptides, may generate an autoimmune response in an immunized animal such that autoantibodies that specifically recognize the animal's endogenous tumor-associated HLA-restricted protein. In specific embodiments, such administration is vaccination with a vaccine comprising PR1. This vaccination technology is shown in U.S. Pat. Nos. 6,027,727; 5,785,970, and 5,609,870, which are hereby incorporated by reference.

As used herein, an “antigenic composition” may comprise an antigen (e.g., a peptide or polypeptide), a nucleic acid encoding an antigen (e.g., an antigen expression vector), or a cell expressing or presenting an antigen. For an antigenic composition, such as a tumor-associated HLA-restricted peptide or antigen of the present invention, to be useful as an immunogenic composition, including a vaccine, the antigenic composition must induce an immune response to the antigen in a cell, tissue or animal (e.g., a human). In particular embodiments, the antigenic composition comprises or encodes all or part of the sequences shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, or an immunologically functional equivalent thereof.

Such peptides should generally be at least five or six amino acid residues in length and will preferably be about 7, 8, 9, 10, 11, 12, 13, 14, 15, 16, 17, 18, 19, 20, 25 or about 30 amino acid residues in length, and may contain up to about 35-50 residues. For example, these peptides may comprise an amino acid sequence, such as 5, 6, 7, 8, and 9 or more contiguous amino acids from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35. Synthetic peptides can generally be about 35 residues long, which is the approximate upper length limit of automated peptide synthesis machines, such as those available from Applied Biosystems (Foster City, Calif.). Longer peptides also may be prepared, e.g., by recombinant means.

In specific embodiments, peptides of the invention may be at least 70%, 75%, 77%, 80%, 82%, 85%, 87%, 90%, 92%, 95%, 97%, or 99% identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, and/or SEQ ID NO:35.

U.S. Pat. No. 4,554,101 (“Hopp”), incorporated herein by reference, teaches the identification and preparation of epitopes from primary amino acid sequences on the basis of hydrophilicity. Through the methods disclosed in Hopp, one of skill in the art would be able to identify epitopes from within an amino acid sequence such as the tumor-associated HLA-restricted peptides sequences disclosed herein in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35.

As used herein, an “amino acid molecule” or “amino acid residue” refers to any naturally occurring amino acid, any amino acid derivative or any amino acid mimic known in the art, including modified or unusual amino acids. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties. In specific aspects, the composition of the present invention employs a peptide of from about 5 to about 100 amino acids or greater in length.

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases located at the National Institutes of Health website. The coding regions for known genes may be amplified and/or expressed using the techniques disclosed herein or as would be know to those of ordinary skill in the art. Alternatively, various commercial preparations of proteins, polypeptides and peptides are known to those of skill in the art.

The term “peptide” is used interchangeably with “oligopeptide” in the present specification to designate a series of residues, typically L-amino acids, connected one to the other, typically by peptide bonds between the α-amino and carboxyl groups of adjacent amino acids. The particular CTL-inducing oligopeptides of the invention are 13 residues or less in length and usually consist of between about 8 and about 11 residues, particularly 9 or 10 residues. The particular HTL-inducing oligopeptides are less than about 50 residues in length and usually consist of between about 6 and about 30 residues, more usually between about 12 and 25, and often between about 15 and 20 residues.

In certain embodiments the size of the at least one peptide molecule may comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, or greater amino molecule residues, and any range derivable therein.

An “immunogenic peptide” or “peptide epitope” is a peptide which comprises an allele-specific motif or supermotif such that the peptide will bind an HLA molecule and induce a CTL and/or HTL response. Thus, immunogenic peptides of the invention are capable of binding to an appropriate HLA molecule and thereafter inducing a cytotoxic T cell response, or a helper T cell response, to the antigen from which the immunogenic peptide is derived.

Accordingly, the term “proteinaceous composition” encompasses amino molecule sequences comprising at least one of the 20 common amino acids in naturally synthesized proteins, or at least one modified or unusual amino acid, including but not limited to those shown on Table 1 below.

TABLE 1
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | 2-alanine, -Amino-pro- | Ahyl | Allo-Hydroxylysine |
| | pionic acid | 3Hyp | 3-Hydroxyproline |
| Abu | 2-Aminobutyric acid | 4Hyp | 4-Hydroxyproline |
| 4Abu | 4-Aminobutyric acid, | Ide | Isodesmosine |
| | piperidinic acid | Aile | Allo-Isoleucine |
| Acp | 6-Aminocaproic acid | MeGly | N-Methylglycine, |
| Ahe | 2-Aminoheptanoic acid | | sarcosine |
| Aib | 2-Aminoisobutyric acid | MeIle | N-Methylisoleucine |
| Baib | 3-Aminoisobutyric acid | MeLys | 6-N-Methyllysine |
| Apm | 2-Aminopimelic acid | MeVal | N-Methylvaline |
| Dbu | 2,4-Diaminobutyric acid | Nva | Norvaline |
| Des | Desmosine | Nle | Norleucine |
| Dpm | 2,2′-Diaminopimelic acid | Orn | Ornithine |
| Dpr | 2,3-Diaminopropionic acid | | |
| EtGly | N-Ethylglycine | | |

In certain embodiments, the proteinaceous composition comprises at least one protein, polypeptide or peptide. In further embodiments, the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

In certain embodiments, the proteinaceous composition may comprise at least one antibody, for example, an antibody against PR1 or myeloperoxidase. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Harlow et al., 1988; incorporated herein by reference).

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

A. Fusion Proteins of HLA-Restricted Peptides

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

B. Variants of HLA-Restricted Peptides

It is contemplated that peptides of the present invention may further employ amino acid sequence variants such as substitutional, insertional or deletion variants. Deletion variants lack one or more residues of the native protein. Insertional mutants typically involve the addition of material at a non-terminal point in the polypeptide. Substitutions are changes to an existing amino acid. These sequence variants may generate truncations, point mutations, and frameshift mutations. As is known to one skilled in the art, synthetic peptides can be generated by these mutations.

It also will be understood that amino acids sequence variants may include additional residues, such as additional N- or C-terminal amino acids, and yet still be essentially as set forth in one of the sequences disclosed herein, so long as the sequence meets the criteria set forth above, including the maintenance of biological activity.

The following is a discussion based upon changing the amino acids of a protein, such as a HLA-restricted peptide or protein of the invention, to create a mutated, truncated, or modified protein. For example, certain amino acids may be substituted for other amino acids in the tumor-associated HLA-restricted peptide or protein such as a Pr3 or MYO protein, resulting in a greater CTL immune response in cells such as a myeloid cell. Since it is the interactive capacity and nature of a protein that defines that protein's biological functional activity, certain amino acid substitutions can be made in a protein sequence, and in its underlying nucleic acid coding sequence, thereby producing a mutated, truncated or modified protein.

In specific embodiments, there are modifications to PR1 (VLQELNVTV; SEQ ID NO:1) as it is used in compositions and methods of the invention. For example, there may be amino acid substitutions at any position of PR1 including (numbered from the N-terminal end) position 1, position 2, position 3, position 4, position 5, position 6, position 7, position 8, or position 9, for example. In some embodiments, one or more additional amino acids are added onto the N-terminal end and/or C-terminal end of PR1. In particular embodiments, one or more amino acids of PR1 are substituted and one or more additional amino acids are added onto the N-terminal end and/or C-terminal end of PR1. In specific cases, the amino acid at position 2 or position 9 are not substituted, and in some cases an amino acid is not substituted with proline. In a specific embodiment, a valine is added to the C-terminus of PR1.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present invention may also employ the use of peptide mimetics for the preparation of polypeptides (see e.g., Johnson, 1993) having many of the natural properties of a tumor-associated HLA-restricted peptide such as Pr3 or MYO protein, but with altered and/or improved characteristics. The underlying rationale behind the use of peptide mimetics is that the peptide backbone of proteins exists chiefly to orient amino acid side chains in such a way as to facilitate molecular interactions, such as those of antibody and antigen. These principles may be used, in conjunction with the principles outline above, to engineer second generation molecules having many of the natural properties of a tumor-associated HLA-restricted peptide but with altered and even improved characteristics.

Also, numerous scientific publications have also been devoted to the prediction of secondary structure, and to the identification of epitopes, from analyses of amino acid sequences (Chou and Fasman, 1974a, b; 1978a, b; 1979). Any of these may be used, if desired, to supplement the teachings of Hopp in U.S. Pat. No. 4,554,101.

Moreover, computer programs are currently available to assist with predicting antigenic portions and epitopic core regions of proteins. Examples include those programs based upon the Jameson-Wolf analysis (Jameson and Wolf, 1988; Wolf et al., 1988), the program PepPlot® (Brutlag et al., 1990; Weinberger et al., 1985), and other new programs for protein tertiary structure prediction (Fetrow and Bryant, 1993). Another commercially available software program capable of carrying out such analyses is MacVector (IBI, New Haven, Conn.).

In further embodiments, major antigenic determinants of a tumor-associated HLA-restricted peptide may be identified by an empirical approach in which portions of the gene encoding the tumor-associated HLA-restricted peptides are expressed in a recombinant host, and the resulting proteins tested for their ability to elicit an immune response. For example, PCR™ can be used to prepare a range of peptides lacking successively longer fragments of the C-terminus of the protein. The immunoactivity of each of these peptides is determined to identify those fragments or domains of the polypeptide that are immunodominant. Further studies in which only a small number of amino acids are removed at each iteration then allows the location of the antigenic determinants of the polypeptide to be more precisely determined.

Another method for determining the major antigenic determinants of a polypeptide is the SPOTs system (Genosys Biotechnologies, Inc., The Woodlands, Tex.). In this method, overlapping peptides are synthesized on a cellulose membrane, which following synthesis and deprotection, is screened using a polyclonal or monoclonal antibody. The antigenic determinants of the peptides which are initially identified can be further localized by performing subsequent syntheses of smaller peptides with larger overlaps, and by eventually replacing individual amino acids at each position along the immunoreactive peptide.

Once one or more such analyses are completed, polypeptides are prepared that contain at least the essential features of one or more antigenic determinants. The peptides are then employed in the generation of antisera against the polypeptide. Minigenes or gene fusions encoding these determinants also can be constructed and inserted into expression vectors by standard methods, for example, using PCR™ cloning methodology.

C. Tumor-Associated HLA-Restricted Peptide Purification

In certain embodiments the protein(s) of the present invention may be purified. It may be desirable to purify the tumor-associated HLA-restricted peptides, polypeptides or proteins or variants thereof. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying a tumor-associated HLA-restricted peptide of the present invention, it may be desirable to express the polypeptide in a prokaryotic or eukaryotic expression system and extract the protein using denaturing conditions. The polypeptide may be purified from other cellular components using an affinity column, which binds to a tagged portion of the polypeptide. Although this preparation will be purified in an inactive form, the denatured material will still be capable of transducing cells. Once inside of the target cell or tissue, it is generally accepted that the polypeptide will regain full biological activity.

As is generally known in the art, it is believed that the order of conducting the various purification steps may be changed, or that certain steps may be omitted, and still result in a suitable method for the preparation of a substantially purified protein or peptide.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity, herein assessed by a “− fold purification number.” The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### VIII. IMMUNOGENIC COMPOSITION COMPONENTS

In some embodiments of the invention, the immunogenic composition, such as a tumor-associated HLA-restricted peptide or antigen, comprises an additional immunostimulatory agent or nucleic acids encoding such an agent. Immunostimulatory agents include but are not limited to an additional antigen, an immunomodulator, an antigen presenting cell or an adjuvant, for example. In other embodiments, one or more of the additional agent(s) is covalently bonded to the antigen or an immunostimulatory agent, in any combination. In certain embodiments, the antigenic composition is conjugated to or comprises HLA anchor motif amino acids.

The use of such small peptides for antibody generation or vaccination typically requires conjugation of the peptide to an immunogenic carrier protein, such as hepatitis B surface antigen, keyhole limpet hemocyanin or bovine serum albumin, or other adjuvants discussed above (adjuvenated peptide). Alum is an adjuvant that has proven sufficiently non-toxic for use in humans. Methods for performing this conjugation are well known in the art. Other immunopotentiating compounds are also contemplated for use with the compositions of the invention such as polysaccharides, including chitosan, which is described in U.S. Pat. No. 5,980,912, hereby incorporated by reference. Multiple (more than one) tumor-associated HLA-restricted epitopes may be crosslinked to one another (e.g., polymerized). Alternatively, a nucleic acid sequence encoding a tumor-associated HLA-restricted peptides, or polypeptides may be combined with a nucleic acid sequence that heightens the immune response. Such fusion proteins may comprise part or all of a foreign (non-self) protein such as bacterial sequences, for example.

Antibody titers effective to achieve a response against endogenous tumor-associated HLA-restricted peptides, or polypeptides will vary with the species of the vaccinated animal, as well as with the sequence of the administered peptide. However, effective titers may be readily determined, for example, by testing a panel of animals with varying doses of the specific antigen and measuring the induced titers of autoantibodies (or anti-self antibodies) by known techniques, such as ELISA assays, and then correlating the titers with a related cancer characteristics, e.g., tumor growth or size.

One of ordinary skill would know various assays to determine whether an immune response against a tumor-associated HLA-restricted peptide was generated. The phrase “immune response” includes both cellular and humoral immune responses. Various B lymphocyte and T lymphocyte assays are well known, such as ELISAs, cytotoxic T lymphocyte (CTL) assays, such as chromium release assays, proliferation assays using peripheral blood lymphocytes (PBL), tetramer assays, and cytokine production assays. See Benjamini et al. (1991), hereby incorporated by reference.

A. Adjuvants

As also well known in the art, the immunogenicity of a particular immunogen composition can be enhanced by the use of non-specific stimulators of the immune response, known as adjuvants. Adjuvants have been used experimentally to promote a generalized increase in immunity against unknown antigens (e.g., U.S. Pat. No. 4,877,611). Immunization protocols have used adjuvants to stimulate responses for many years, and as such adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation. For many cancers, there is compelling evidence that the immune system participates in host defense against the tumor cells, but only a fraction of the likely total number of tumor-specific antigens are believed to have been identified to date. The use of the tumor-associated HLA-restricted antigens of the present invention with the inclusion of a suitable adjuvant will likely increase the anti-tumor response of the antigens. Suitable molecule adjuvants include all acceptable immunostimulatory compounds, such as cytokines, toxins or synthetic compositions.

Exemplary adjuvants include complete Freund's adjuvant (a non-specific stimulator of the immune response containing killed Mycobacterium tuberculosis), incomplete Freund's adjuvants, and aluminum hydroxide adjuvant. Other adjuvants that may also be used include IL-1, IL-2, IL-4, IL-7, IL-12, α-interferon, β-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used.

In one aspect, an adjuvant effect is achieved by use of an agent, such as alum, used in about 0.05 to about 0.1% solution in phosphate buffered saline. Alternatively, the antigen is made as an admixture with synthetic polymers of sugars (Carbopol®) used as an about 0.25% solution. Adjuvant effect may also be made by aggregation of the antigen in the vaccine by heat treatment with temperatures ranging between about 70° to about 101° C. for a 30 second to 2-minute period, respectively. Aggregation by reactivating with pepsin treated (Fab) antibodies to albumin, mixture with bacterial cell(s) such as C. parvum, an endotoxin or a lipopolysaccharide component of Gram-negative bacteria, emulsion in physiologically acceptable oil vehicles, such as mannide mono-oleate (Aracel A), or emulsion with a 20% solution of a perfluorocarbon (Fluosol-DA®) used as a block substitute, also may be employed.

With some adjuvants, for example, certain organic molecules obtained from bacteria act on the host rather than on the antigen. An example is muramyl dipeptide (N-acetylmuramyl-L-alanyl-D-isoglutamine [MDP]), a bacterial peptidoglycan. The effects of MDP, as with most adjuvants, are not fully understood. MDP stimulates macrophages but also appears to stimulate B cells directly. The effects of adjuvants, therefore, are not antigen-specific. If they are administered together with a purified antigen, however, they can be used to selectively promote the response to the antigen.

In certain embodiments, hemocyanins and hemoerythrins may also be used in the invention. The use of hemocyanin from keyhole limpet (KLH) is preferred in certain embodiments, although other molluscan and arthropod hemocyanins and hemoerythrins may be employed.

Various polysaccharide adjuvants may also be used. For example, the use of various pneumococcal polysaccharide adjuvants on the antibody responses of mice has been described (Yin et al., 1989). The doses that produce optimal responses, or that otherwise do not produce suppression, should be employed as indicated (Yin et al., 1989). Polyamine varieties of polysaccharides are particularly preferred, such as chitin and chitosan, including deacetylated chitin.

Another group of adjuvants are the muramyl dipeptide (MDP, N-acetylmuramyl-L-alanyl-D-isoglutamine) group of bacterial peptidoglycans. Derivatives of muramyl dipeptide, such as the amino acid derivative threonyl-MDP, and the fatty acid derivative MTPPE, are also contemplated.

U.S. Pat. No. 4,950,645 describes a lipophilic disaccharide-tripeptide derivative of muramyl dipeptide, which is described for use in artificial liposomes formed from phosphatidyl choline and phosphatidyl glycerol. It is to be effective in activating human monocytes and destroying tumor cells, but is non-toxic in generally high doses. The compounds of U.S. Pat. No. 4,950,645 and PCT Patent Application WO 91/16347 are contemplated for use with cellular carriers and other embodiments of the present invention.

BCG (bacillus Calmette-Guerin, an attenuated strain of Mycobacterium) and BCG-cell wall skeleton (CWS) may also be used as adjuvants, with or without trehalose dimycolate. Trehalose dimycolate may be used itself. Trehalose dimycolate administration has been shown to correlate with augmented resistance to influenza virus infection in mice (Azuma et al., 1988). Trehalose dimycolate may be prepared as described in U.S. Pat. No. 4,579,945. BCG is an important clinical tool because of its immunostimulatory properties. BCG acts to stimulate the reticulo-endothelial system, activates natural killer cells and increases proliferation of hematopoietic stem cells. Cell wall extracts of BCG have proven to have excellent immune adjuvant activity. Molecular genetic tools and methods for mycobacteria have provided the means to introduce foreign genes into BCG (Jacobs et al., 1987; Snapper et al., 1988; Husson et al., 1990; Martin et al., 1990). Live BCG is an effective and safe vaccine used worldwide to prevent tuberculosis. BCG and other mycobacteria are highly effective adjuvants, and the immune response to mycobacteria has been studied extensively. With nearly 2 billion immunizations, BCG has a long record of safe use in man (Luelmo, 1982; Lotte et al., 1984). It is one of the few vaccines that can be given at birth, it engenders long-lived immune responses with only a single dose, and there is a worldwide distribution network with experience in BCG vaccination. An exemplary BCG vaccine is sold as TICE BCG (Organon Inc., West Orange, N.J.).

Amphipathic and surface active agents, e.g., saponin and derivatives such as QS21 (Cambridge Biotech), form yet another group of adjuvants for use with the immunogens of the present invention. Nonionic block copolymer surfactants (Rabinovich et al., 1994) may also be employed. Oligonucleotides are another useful group of adjuvants (Yamamoto et al., 1988). Quil A and lentinen are other adjuvants that may be used in certain embodiments of the present invention.

Another group of adjuvants are the detoxified endotoxins, such as the refined detoxified endotoxin of U.S. Pat. No. 4,866,034. These refined detoxified endotoxins are effective in producing adjuvant responses in mammals. Of course, the detoxified endotoxins may be combined with other adjuvants to prepare multi-adjuvant-incorporated cells. For example, combination of detoxified endotoxins with trehalose dimycolate is particularly contemplated, as described in U.S. Pat. No. 4,435,386. Combinations of detoxified endotoxins with trehalose dimycolate and endotoxic glycolipids is also contemplated (U.S. Pat. No. 4,505,899), as is combination of detoxified endotoxins with cell wall skeleton (CWS) or CWS and trehalose dimycolate, as described in U.S. Pat. Nos. 4,436,727, 4,436,728 and 4,505,900. Combinations of just CWS and trehalose dimycolate, without detoxified endotoxins, is also envisioned to be useful, as described in U.S. Pat. No. 4,520,019.

Those of skill in the art will know the different kinds of adjuvants that can be conjugated to cellular vaccines in accordance with this invention and these include alkyl lysophosphilipids (ALP); BCG; and biotin (including biotinylated derivatives) among others. Certain adjuvants particularly contemplated for use are the teichoic acids from Gram-cells. These include the lipoteichoic acids (LTA), ribitol teichoic acids (RTA) and glycerol teichoic acid (GTA). Active forms of their synthetic counterparts may also be employed in connection with the invention (Takada et al., 1995).

Various adjuvants, even those that are not commonly used in humans, may still be employed in animals, where, for example, one desires to raise antibodies or to subsequently obtain activated T cells. The toxicity or other adverse effects that may result from either the adjuvant or the cells, e.g., as may occur using non-irradiated tumor cells, is irrelevant in such circumstances.

Adjuvants may be encoded by a nucleic acid (e.g., DNA or RNA). It is contemplated that such adjuvants may be also be encoded in a nucleic acid (e.g., an expression vector) encoding the antigen, or in a separate vector or other construct. Nucleic acids encoding the adjuvants can be delivered directly, such as for example with lipids or liposomes.

B. Biological Response Modifiers

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m²) (Johnson/Mead, NJ), cytokines such as -interferon, IL-2, or IL-12 or genes encoding proteins involved in immune helper functions, such as B-7.

C. Chemokines

Chemokines, nucleic acids that encode for chemokines, and/or cells that express such also may be used as vaccine components. Chemokines generally act as chemoattractants to recruit immune effector cells to the site of chemokine expression. It may be advantageous to express a particular chemokine coding sequence in combination with, for example, a cytokine coding sequence, to enhance the recruitment of other immune system components to the site of treatment. Such chemokines include, for example, RANTES, MCAF, MIP1-alpha, MIP1-Beta, IP-10 and combinations thereof. The skilled artisan will recognize that certain cytokines are also known to have chemoattractant effects and could also be classified under the term chemokines.

D. Immunogenic Carrier Proteins

In certain embodiments, an antigenic composition may be chemically coupled to a carrier or recombinantly expressed with a immunogenic carrier peptide or polypeptide (e.g., a antigen-carrier fusion peptide or polypeptide) to enhance an immune reaction. Exemplary and preferred immunogenic carrier amino acid sequences include hepatitis B surface antigen, keyhole limpet hemocyanin (KLH) and bovine serum albumin (BSA). Other albumins such as ovalbumin, mouse serum albumin or rabbit serum albumin also can be used as immunogenic carrier proteins. Means for conjugating a polypeptide or peptide to a immunogenic carrier protein are well known in the art and include, for example, glutaraldehyde, m-maleimidobenzoyl-N-hydroxysuccinimide ester, carbodiimide and bis-biazotized benzidine.

### IX. ANTIBODIES AND ANTIBODY GENERATION

Another embodiment of the present invention are antibodies, in some cases, a human monoclonal antibody immunoreactive with the polypeptide sequence of a tumor-associated HLA-restricted peptide of the invention comprising SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35. It is also understood that this antibody is useful for screening samples from human patients for the purpose of detecting a particular tumor-associated HLA-restricted peptide present in the samples. The antibody also may be useful in the screening of expressed DNA segments or peptides and proteins for the discovery of related antigenic sequences. In addition, the antibody may be useful in passive immunotherapy for cancer. All such uses of the antibodies and any antigens or epitopic sequences so discovered fall within the scope of the present invention.

Examples of other antibodies that may be employed in the present invention may include antibodies that react with T cells such as CD1, CD2, CD3, CD5, CD7 CD4, CD6, CD8 and CD27. Antibodies that react with myeloid cells may also be employed and include CD11b, CD11c, CD13, CD14, CD15, CD16, CD33, CD48, CD63, CD74, CD65, CD66, CD67 and CD68. Antibodies that react with undifferentiated cells may include HLA-DR, CD34 and CD38. It should be appreciated that multiple combinations of antibodies selected from the ones mentioned above are possible. Accordingly, it will be apparent to one skilled in the art that one can vary the antibody combinations

In certain embodiments, the present invention involves antibodies. For example, all or part of a monoclonal, single chain, or humanized antibody may function as a vaccine for cancer. Other aspects of the invention involve administering antibodies as a form of treatment or as a diagnostic to identify or quantify a particular polypeptide, such as tumor-associated HLA-restricted polypeptide, for example Pr3 or MYO polypeptide. As detailed above, in addition to antibodies generated against full length proteins, antibodies also may be generated in response to smaller constructs comprising epitopic core regions, including wild-type and mutant epitopes.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is may also be used to refer to any antibody-like molecule that has an antigen binding region, and includes antibody fragments such as Fab′, Fab, F(ab′)2, single domain antibodies (DABs), Fv, scFv (single chain Fv), and the like. The techniques for preparing and using various antibody-based constructs and fragments are well known in the art. Means for preparing and characterizing antibodies are also well known in the art (See, Harlow and Lane, 1988; incorporated herein by reference).

Monoclonal antibodies (mAbs) are recognized to have certain advantages, e.g., reproducibility and large-scale production, and their use is generally preferred. The invention thus provides monoclonal antibodies of the human, murine, monkey, rat, hamster, rabbit and even chicken origin.

The methods for generating monoclonal antibodies (mAbs) generally begin along the same lines as those for preparing polyclonal antibodies. Briefly, a polyclonal antibody may be prepared by immunizing an animal with an immunogenic polypeptide composition in accordance with the present invention and collecting antisera from that immunized animal. Alternatively, in some embodiments of the present invention, serum is collected from persons who may have been exposed to a particular antigen. Exposure to a particular antigen may occur a work environment, such that those persons have been occupationally exposed to a particular antigen and have developed polyclonal antibodies to a peptide, polypeptide, or protein. In some embodiments of the invention polyclonal serum from occupationally exposed persons is used to identify antigenic regions in the gelonin toxin through the use of immunodetection methods.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin also can be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection also may be given. The process of boosting and tittering is repeated until a suitable titer is achieved. When a desired level of immunogenicity is obtained, the immunized animal can be bled and the serum isolated and stored, and/or the animal can be used to generate mAbs.

mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified polypeptide, peptide or domain, be it a wild-type or mutant composition. The immunizing composition is administered in a manner effective to stimulate antibody producing cells.

mAbs may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate mAbs. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Humanized monoclonal antibodies are antibodies of animal origin that have been modified using genetic engineering techniques to replace constant region and/or variable region framework sequences with human sequences, while retaining the original antigen specificity. Such antibodies are commonly derived from rodent antibodies with specificity against human antigens. Such antibodies are generally useful for in vivo therapeutic applications. This strategy reduces the host response to the foreign antibody and allows selection of the human effector functions.

“Humanized” antibodies are also contemplated, as are chimeric antibodies from mouse, rat, or other species, bearing human constant and/or variable region domains, bispecific antibodies, recombinant and engineered antibodies and fragments thereof. The techniques for producing humanized immunoglobulins are well known to those of skill in the art. For example U.S. Pat. No. 5,693,762 discloses methods for producing, and compositions of, humanized immunoglobulins having one or more complementarity determining regions (CDR's). When combined into an intact antibody, the humanized immunoglobulins are substantially non-immunogenic in humans and retain substantially the same affinity as the donor immunoglobulin to the antigen, such as a protein or other compound containing an epitope. Examples of other teachings in this area include U.S. Pat. Nos. 6,054,297; 5,861,155; and 6,020,192, all specifically incorporated by reference. Methods for the development of antibodies that are “custom-tailored” to the patient's disease are likewise known and such custom-tailored antibodies are also contemplated.

### X. NUCLEIC ACIDS ENCODING HLA-RESTRICTED PROTEIN, PEPTIDES AND POLYPEPTIDES

It is contemplated in the present invention, that the tumor-associated HLA-restricted peptides, or polypeptides may be encoded by a nucleic acid sequence. A nucleic acid may be derived from genomic DNA, complementary DNA (cDNA) or synthetic DNA. Where incorporation into an expression vector is desired, the nucleic acid may also comprise a natural intron or an intron derived from another gene.

As used herein, the term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy. A tumor-associated HLA-restricted peptide or polypeptide cDNA, such as a Pr3 or MYO cDNA, for use in the present invention, may be derived from human cDNA but are not limited such.

As used herein, the term “nucleic acid segment” refers to a nucleic acid molecule that has been isolated free of total genomic DNA of a particular species. Therefore, a nucleic acid segment encoding a polypeptide refers to a nucleic acid segment that contains wild-type, polymorphic, or mutant polypeptide-coding sequences yet is isolated away from, or purified free from, total mammalian or human genomic DNA. Included within the term “nucleic acid segment” are a polypeptide or polypeptides, DNA segments smaller than a polypeptide, and recombinant vectors, such as, plasmids and other non-viral vectors.

The term “recombinant” may be used in conjunction with a polypeptide or the name of a specific polypeptide, and generally refers to a polypeptide produced from a nucleic acid molecule that has been manipulated in vitro or that is the replicated product of such a molecule. Recombinant vectors and isolated nucleic acid segments may variously include the PR1 or myeloperoxidase-coding regions themselves, coding regions bearing selected alterations or modifications in the basic coding region, or they may encode larger polypeptides that nevertheless include PR1 or myeloperoxidase-coding regions or may encode biologically functional equivalent proteins or peptides that have variant amino acids sequences.

A “nucleic acid” as used herein includes single-stranded and double-stranded molecules, as well as DNA, RNA, chemically modified nucleic acids and nucleic acid analogs. It is contemplated that a nucleic acid within the scope of the present invention may be of about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater nucleotide residues in length. Those of skill will recognize that in cases where the nucleic acid region encodes a tumor-associated HLA-restricted peptide, or polypeptide, the nucleic acid region can be quite long, depending upon the number of amino acids in the fusion protein.

It is contemplated that the tumor-associated HLA-restricted peptide, or polypeptide may be encoded by any nucleic acid sequence that encodes the appropriate amino acid sequence. The design and production of nucleic acids encoding a desired amino acid sequence is well known to those of skill in the art, using standardized codon tables (Table 2). In preferred embodiments, the codons selected for encoding each amino acid may be modified to optimize expression of the nucleic acid in the host cell of interest. The term “functionally equivalent codon” is used herein to refer to codons that encode the same amino acid, such as the six codons for arginine or serine, and also refers to codons that encode biologically equivalent amino acids. Codon preferences for various species of host cell are well known in the art. Codons preferred for use in humans, are well known to those of skill in the art (Wada et. al., 1990). Codon preferences for other organisms also are well known to those of skill in the art (Wada et al., 1990, included herein in its entirety by reference)

TABLE 2
Codon Table

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Prokaryote- and/or eukaryote-based systems can be used to produce nucleic acid sequences, or their cognate polypeptides, proteins and peptides. The present invention contemplates the use of such an expression system to produce the tumor-associated HLA-restricted peptide, or polypeptide. More specifically, the present invention employs the use of the insect cell/baculovirus system. The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MaxBac® 2.0 from Invitrogen® and BacPack™ Baculovirus Expression System From Clontech®.

In addition to the expression system disclosed in the invention, numerous expression systems exists which are commercially and widely available. One example of such a system is the Stratagene®'s Complete Control Inducible Mammalian Expression System, which involves a synthetic ecdysone-inducible receptor, or its pET Expression System, an E. coli expression system. Another example of an inducible expression system is available from Invitrogen®, which carries the T-Rex™ (tetracycline-regulated expression) System, an inducible mammalian expression system that uses the full-length CMV promoter. Invitrogen® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### XI. VIRAL VECTORS

There are a number of ways in which expression vectors may be introduced into cells. In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986).

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the vaccines of the present invention (Muzyczka, 1992). AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses have promise as gene delivery vectors in vaccines due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid (e.g., one encoding an antigen of interest) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

B. Nucleic Acid Delivery

Suitable methods for nucleic acid delivery to effect expression of compositions of the present invention are believed to include virtually any method by which a nucleic acid (e.g., DNA, including viral and nonviral vectors) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

### XII. PHARMACEUTICAL VACCINE COMPOSITIONS, DELIVERY, AND TREATMENT REGIMENS

In an embodiment of the present invention, a method of treatment and prevention of breast cancers by the delivery of a tumor-associated HLA-restricted PR1 peptide or expression construct is contemplated. An effective amount of the pharmaceutical vaccine composition, generally, is defined as that amount sufficient to detectably and repeatedly to ameliorate, reduce, minimize or limit the extent of the disease or condition or symptoms thereof. More rigorous definitions may apply, including elimination, eradication or cure of disease. Preferably, patients will have adequate bone marrow function (defined as a peripheral absolute granulocyte count of >2,000/mm³ and a platelet count of 100,000/mm³), adequate liver function (bilirubin <1.5 mg/dl) and adequate renal function (creatinine <1.5 mg/dl).

A. HLA-Restricted Vaccine Administration

To kill cells, inhibit cell growth, inhibit metastasis, decrease tumor or tissue size and otherwise reverse or reduce the malignant phenotype of tumor cells, using the methods and compositions of the present invention, one would generally contact a cancer cell with the therapeutic compound such as a polypeptide or an expression construct encoding a polypeptide. The routes of administration will vary, naturally, with the location and nature of the lesion, and include, e.g., intradermal, transdermal, parenteral, intravenous, intramuscular, intranasal, subcutaneous, percutaneous, intratracheal, intraperitoneal, intratumoral, perfusion, lavage, direct injection, and oral administration and formulation. Any of the formulations and routes of administration discussed with respect to the treatment or diagnosis of cancer may also be employed with respect to neoplastic diseases and conditions.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (preferably 10 ml), while for tumors of <4 cm, a volume of about 1-3 ml will be used (preferably 3 ml). Multiple injections delivered as single dose comprise about 0.1 to about 0.5 ml volumes. The viral particles may advantageously be contacted by administering multiple injections to the tumor, spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising a tumor-associated HLA restricted peptide or construct encoding therefor. The perfusion may be continued post-resection, for example, by leaving a catheter implanted at the site of the surgery. Periodic post-surgical treatment also is envisioned.

Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hr, to about 2-6 hr, to about 6-12 hr, to about 12-24 hr, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatment regimens may vary as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) for a viral construct. Unit doses range from 10³, 10⁴, 10⁵, 10⁶, 10⁷, 10⁸, 10⁹, 10¹⁰, 10¹¹, 10¹², 10¹³ pfu and higher. Alternatively, depending on the kind of virus and the titer attainable, one will deliver 1 to 100, 10 to 50, 100-1000, or up to about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ or higher infectious viral particles (vp) to the patient or to the patient's cells.

In a specific embodiment, the immunogenic composition is provided to an individual in need thereof multiple times over the course of several weeks or months. In a particular case, the immunogenic composition is provided 4 times over 3 weeks, followed by an additional dose provided 4 months later.

B. Injectable Compositions and Formulations

One method for the delivery of a pharmaceutical according to the present invention is systemically. However, the pharmaceutical compositions disclosed herein may alternatively be administered parenterally, intravenously, intradermally, intramuscularly, transdermally or even intraperitoneally as described in U.S. Pat. No. 5,543,158; U.S. Pat. No. 5,641,515 and U.S. Pat. No. 5,399,363 (each specifically incorporated herein by reference in its entirety).

Injection of pharmaceuticals may be by syringe or any other method used for injection of a solution, as long as the agent can pass through the particular gauge of needle required for injection. A novel needleless injection system has been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermolysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

### XIII. COMBINATION TREATMENTS

The compounds and methods of the present invention may be used in the context of neoplastic diseases/conditions including breast cancer. In order to increase the effectiveness of a breast cancer treatment with the tumor-associated HLA-restricted compositions of the present invention, such as Pr3 or MYO peptide, polypeptide, protein, or expression construct coding therefor, it may be desirable to combine one or more of these compositions with other agents effective in the treatment of those diseases and conditions. For example, the treatment of a cancer may be implemented with therapeutic compounds of the present invention and other anti-cancer therapies, such as anti-cancer agents or surgery.

Various combinations may be employed; for example, the tumor-associated HLA-restricted peptide is “A” and the secondary anti-cancer is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Administration of the therapeutic agents of the present invention to a patient will follow general protocols for the administration of that particular secondary therapy, taking into account the toxicity, if any, of the tumor-associated HLA-restricted peptide treatment. It is expected that the treatment cycles would be repeated as necessary. It also is contemplated that various standard therapies, as well as surgical intervention, may be applied in combination with the described cancer cell.

A. Adjunct Anti-Cancer Therapy

An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver et al., 1992). In the context of the present invention, it is contemplated that tumor-associated HLA-restricted peptide therapy could be used similarly in conjunction with chemotherapeutic, radiotherapeutic, immunotherapeutic or other biological intervention, in addition to other pro-apoptotic or cell cycle regulating agents.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

1. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The present invention contemplates any chemotherapeutic agent that may be employed or known in the art for treating or preventing cancers.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

3. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of Fortilin would provide therapeutic benefit in the treatment of cancer.

Immunotherapy could also be used as part of a combined therapy. The general approach for combined therapy is discussed below. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as mda-7 has been shown to enhance anti-tumor effects (Ju et al., 2000).

As discussed earlier, examples of immunotherapies currently under investigation or in use are immune adjuvants (e.g., Mycobacterium bovis, Plasmodium falciparum, dinitrochlorobenzene and aromatic compounds) (U.S. Pat. No. 5,801,005; U.S. Pat. No. 5,739,169; Hui and Hashimoto, 1998; Christodoulides et al., 1998), cytokine therapy (e.g., interferons, and; IL-1, GM-CSF and TNF) (Bukowski et al., 1998; Davidson et al., 1998; Hellstrand et al., 1998) gene therapy (e.g., TNF, IL-1, IL-2, p53) (Qin et al., 1998; Austin-Ward and Villaseca, 1998; U.S. Pat. No. 5,830,880 and U.S. Pat. No. 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Combination therapy of cancer with herceptin and chemotherapy has been shown to be more effective than the individual therapies. Thus, it is contemplated that one or more anti-cancer therapies may be employed with the tumor-associated HLA-restricted peptide therapies described herein.

i) Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated antigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

ii) Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient. However, their application is somewhat limited by their scarcity and have so far only been administered intralesionally. Human monoclonal antibodies to ganglioside antigens have been administered intralesionally to patients suffering from cutaneous recurrent melanoma (Irie & Morton, 1986). Regression was observed in six out of ten patients, following, daily or weekly, intralesional injections. In another study, moderate success was achieved from intralesional injections of two human monoclonal antibodies (Irie et al., 1989).

It may be favorable to administer more than one monoclonal antibody directed against two different antigens or even antibodies with multiple antigen specificity. Treatment protocols also may include administration of lymphokines or other immune enhancers as described by Bajorin et al. (1988). The development of human monoclonal antibodies is described in further detail elsewhere in the specification.

iii) Active Immunotherapy

In active immunotherapy, an antigenic peptide, polypeptide or protein, or an autologous or allogenic tumor cell composition or “vaccine” is administered, generally with a distinct bacterial adjuvant (Ravindranath and Mitchell et al., 1990; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

4. Genes

In yet another embodiment, the secondary treatment is a gene therapy in which a therapeutic polynucleotide is administered before, after, or at the same time as the tumor-associated HLA-restricted peptide is administered. Delivery of a vector encoding a the tumor-associated HLA-restricted peptide in conjunction with a second vector encoding one of the following gene products will have a combined anti-hyperproliferative effect on target tissues. Alternatively, a single vector encoding both genes may be used. A variety of proteins are encompassed within the invention, some of which are described below. Various genes that may be targeted for gene therapy of some form in combination with the present invention are will known to one of ordinary skill in the art and may comprise any gene involved in cancers.

i) Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

ii) Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

In addition to p53, which has been described above, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primay uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

iii) Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### XIV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary PR1 and Breast Cancer Embodiments

Data has accumulated over the past 30 years showing that the serine proteases proteinase 3 (P3) and neutrophil elastase (NE) are aberrantly expressed in both primary human breast cancer cells and breast cancer cell lines, but not in normal mammary tissue (Finlay et al., 1993; Sato et al., 2006; Yamashita et al., 1994). NE has been shown to cleave cyclin E into its constitutively active high molecular weight isoforms, and NE expression in breast cancer has been shown to have prognostic significance (Harwell et al., 2000; Akizuki et al., 2007; Desmedt et al., 2006; Foekens et al., 2003; Porter et al., 2001). The inventor has previously analyzed tissue-derived mRNA to show that NE and PRTN3 are not expressed in normal human breast tissue.

PR1, an HLA-A2-restricted peptide, is derived from both P3 and NE, and it is recognized on the surface of myeloid leukemia cells by cytotoxic T lymphocytes (CTL) that preferentially kill leukemia and contribute to cytogenetic remission in CML patients. Preliminary results of a phase I/II PR1 peptide vaccine trial in myeloid leukemia patients show that immune responses and clinical responses can be induced by PR1 vaccination and that vaccine-induced immunity to PR1 correlates with improved overall long-term survival.

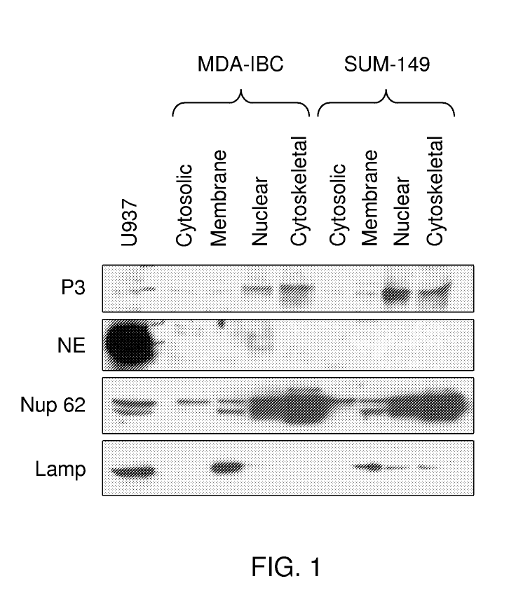

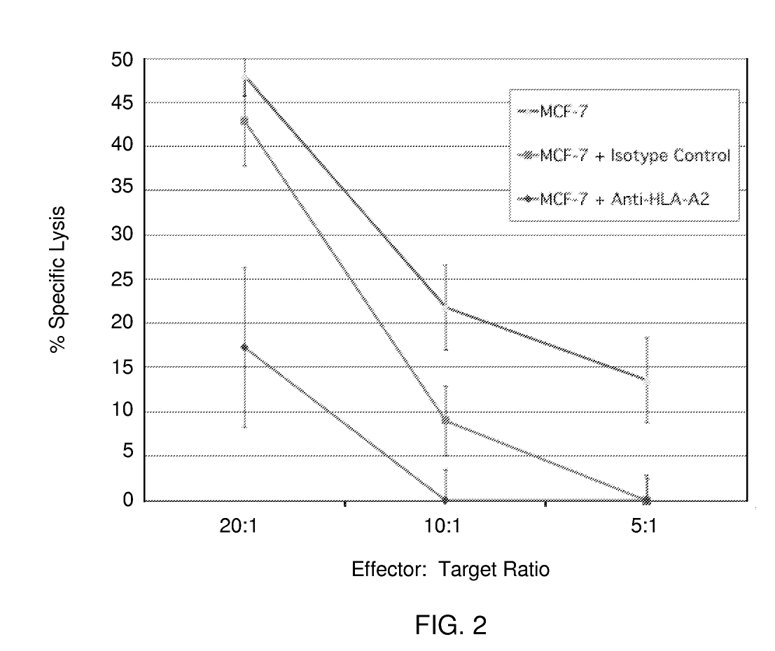

The inventor confirms the protein expression of P3 and NE in three human breast cancer cells (MCF-7, MDA-IBC, SUM-149) by immunoblot of whole cell lysates and of subcellular fractions. The majority of P3 and NE is contained in the cytoplasm and the nucleus instead of lysosomes or endosomes, where they normally reside in neutrophils (FIG. 1). Because PR1-specific CTL specifically lyse leukemia target cells that aberrantly express P3 and NE, in specific embodiments of the invention PR1-CTL would also lyse HLA-A2+ human breast cancer cells that express P3 and NE. PR1-CTL were elicited from an HLA-A2 healthy donor in vitro by weekly stimulations of lymphocytes with PR1-pulsed T2 cells, an HLA-A2+ cell line. After 26 days, the PR1-CTL were incubated with HLA-A2+ MCF-7 cells, which were shown by others to express NE, with or without a blocking antibody to HLA-A2 (BB7.2) versus isotype control antibody. The target cells were labeled with the fluorochrome calcein AM to demonstrate specific lysis after 4 hours co-incubation. Importantly, PR1-CTL induced 47% specific lysis of MCF-7 cells versus only 17% of MCF-7 cells incubated with anti-HLA-A2 blocking antibody (p<0.01) (FIG. 2).

These data validate the PR1 peptide as a target antigen in breast cancer cells and that PR1-specific cellular immunity can result in specific targeting and lysis of breast cancer. Therefore, PR1 is an effective target for use in immunotherapy strategies against breast cancer.

### Example 2

### Exemplary Clinical Trials

Based upon pre-clinical studies, the toxicity and efficacy of PR1 peptide vaccination for patients with breast cancer is investigated. A study is conducted in two phases: a Phase I initial toxicity phase (in order to determine initial vaccine safety), and a Phase II efficacy and toxicity phase.

PR1 peptide is injected subcutaneously in incomplete Freund's adjuvant every 3 weeks for 3 injections to induce a PR1 specific host response against breast cancer. Both in Phase I and in Phase II, patients will also be evaluated for signs of immune reactivity. Before, during, and at the end of the 9 week period of vaccination, the peripheral blood mononuclear cells (PBMC) from the patients will be tested for the development of PR1 immune reactivity in vitro using cytotoxic T lymphocyte precursor frequency (CTLPf) assays against PR1-loaded target cells and against the patient's own breast cancer (a measure of efficacy), PR1/HLA-A2 tetramer staining, 8-color flow cytometry for surface phenotype (memory/naïve, activation), and cytolysis assays of bulk PR1-stimulated PBMC from the patients. The amount of Proteinase 3 expression in the breast cancer cells is studied by cytoplasmic flow cytometry analysis. Any clinical responses (defined by standard criteria as hematological and/or cytogenetic responses) will be correlated with the in vitro testing. Vaccination at 3 dose levels of peptide with a fixed amount of IFA will be conducted, and stopping criteria will be guided by established toxicity criteria.

Phase I. In Phase I of this study, patients are assigned to three escalating peptide dose levels starting with dose level 1. Patients are followed in dose cohorts of size 3 for signs of dose limiting toxicity. Patients are assigned to the next highest dose level cohort only if no more than 1 of the 3 patients at any time at any dose level has ≧grade 3 non-hematological toxicity or autoimmune phenomena (i.e., dose limiting toxicity). At each dose level, the first patient entered must complete two of the three vaccinations prior to initiation of the second patient in that cohort at that dose level. Before the third patient is enrolled, the first patient in each dose cohort must complete all vaccinations and the second patient must complete at least two vaccinations prior to initiation of vaccination in the third patient. Again, if <grade 3 non-hematological toxicity and no autoimmune phenomena are observed in the first three patients, then the next dose level will accrue patients in a similar manner. If more than ⅓ of the patients enrolled at any time at any dose level have grade III or IV non-hematological toxicity, then maximal tolerated dose (MTD) has been exceeded and this and all higher peptide dose levels will be eliminated from Phase II of the study. Dose escalation to the next dose level will occur only after the third patient has been followed for three weeks after the third vaccination in the series and has no dose limiting toxicity.

If an allergic reaction ≧grade 2 occurs in any patient, no further vaccinations will be given in that patient and they will be taken off study. Allergic reactions will be treated with solumedrol 1 mg/kg IV bolus, benadryl 50 mg IV bolus, and epinephrine 0.5 mg S.C.

Phase II. Phase II of the study will be conducted according to a continuous reassessment statistical model. Patients will be randomized among three dose levels, with a maximum of 20 patients per dose level. Only those dose levels without dose limiting toxicity as determined in Phase I of this study will be examined in Phase II. Both toxicity and efficacy will be determined as primary endpoints. Patients will be monitored in cohorts of size 4, and all patients in any cohort will be observed for at least 2 weeks from the first dose of the last patient in the group in the absence of grade III or IV non-hematological toxicity before continuing to the next cohort. Toxicity information will be carefully accrued using established procedures. If grade III or IV non-hematological toxicity is observed during the 9 week study period in 2 of the first 4 patients (the first cohort), 3 of the first 8, 4 of the first 12, or 5 of the first 16 in any of the three dose groups, then that dose level will be terminated and patients will be treated only on the remaining dose levels. Moving to the next cohort will occur only if dose-limiting toxicity is not reached in the number of patients defined above for each cohort.

There will be a two-week observation period before the next vaccination will be given in any patient that experiences grade II non-hematological toxicity <dose limiting toxicity. If the toxicity decreases to grade I or less within those two weeks, the patient will be given the next vaccination. If the grade II toxicity does not decrease to grade I or less within two weeks, the patient will be taken off protocol (removed from study).

Any grade toxicity that provides clear evidence of an autoimmune reaction will be considered a dose limiting toxicity. Such a reaction will preclude further administration of peptide under this protocol, and the study will be terminated. If there is evidence that vaccine administration has produced a Wegener's-like vasculitis or inflammatory disease, then the study will be terminated.

Efficacy, defined as an immune response to PR1 vaccine, will also be determined as a primary endpoint of the study. If none of the first 12 patients have an immune response at a particular dose level, then that dose level will be terminated. Patients will not be retreated in this protocol after the required 3 immunizations, nor will the dose be escalated beyond 1.0 mg of PR1 peptide.

The maximal tolerated dose (MTD) is defined as the highest peptide dose that does not cause dose-limiting non-hematological toxicity beyond the allowable number of patients stated for each phase at each dose level cohort (dose limiting toxicity). The MTD will be determined in either Phase I or Phase II of this study if: (1) dose-limiting toxicity is reached in more than 1 patient of 3 at each dose level cohort in Phase I, or (2) if dose-limiting non-hematological toxicity is exceeded in 3 of the first 8, 4 of the first 12, or 5 of the first 16 in any of the three dose groups in Phase II.

### Example 3

### Illustrative and Exemplary Leukemia Embodiments

As further provided below, in these Examples, there is disclosure for PR1 embodiments employed for immunity to leukemia. The skilled artisan, based on the teachings in Example 1 and with the following Examples as a guideline, can utilize the following description in characterizing PR1 and other peptides for breast cancer treatment and/or prevention.

### Generation of PR1-CTL and Ex Vivo Studies

Methodology. PR1 peptide was combined into the binding region of the HLA-A2 heavy chain, and the resulting protein folded with β2-microglobulin and attached the resulting PR1/HLA-A2 monomer onto 50 nm magnetic microbeads (Miltenyi Co.) (Wang et al., 2000). To do this, the technology used was adapted to assemble PR1/HLA-A2 tetramers, where heavy chain monomers are biotinylated at the C terminus and combined in a 4:1 molar ratio with streptavidin, which has in turn been conjugated to phycoerythrin (PE). The PR1 monomer-conjugated microbeads can then be passed through a sterile column that is surrounded by a magnet that traps microbead-adherent T cells. After the non-adherent cells pass through the column, the column is removed from the magnet and the PR1-specific T cells attached to the microbeads can be collected. This method allows for the selection of PR1-specific T cells, which could be further expanded and given to patients with breast cancer to facilitate graft-versus-breast cancer without graft-versus-host-disease (GVHD).

### Preliminary Studies

Binding assays. Peptide binding to HLA-A2.1 was confirmed using two assays. In the first, indirect flow cytometry was used to measure HLA-A2.1 surface expression on the A2+ T2 cell line coated with the peptide. T2 cells are a human lymphocyte line that lacks TAP1 and TAP2 genes and therefore cannot present endogenous MHC class I restricted antigens. If the peptide effectively bound HLA-A2.1, it stabilized the complex with (β2-microglobulin and increased HLA-A2.1 surface expression, which could be measured using flow cytometry. An HLA-A2.1 specific monoclonal antibody (BB7.2, ATCC, Rockville, Md.) followed by a FITC-labeled secondary antibody (CalTag Laboratories, Burlingame, Calif.) was used to measure surface expression of HLA-A2.1.

In the second assay, the dissociation rate of I¹²⁵-labeled (β2-microglobulin from the heterotrimer complex of the HLA-A2.1 heavy chain, peptide, and β2-microglobulin was measured, which allowed calculation of binding half-life (T_{1/2}). The labeled heterotrimer complex was separated from unincorporated (β2-microglobulin by high-performance liquid chromatography gel filtration, and the half-time of dissociation of β2-microglobulin was determined by subjecting aliquots of the complex to a second round of gel filtration.

PR1 showed increased surface HLA-A2.1 expression compared with T2 cells with no added peptide, as the background for HLA-A2.1 expression. The long measured T_{1/2} as measured using (β2-microglobulin dissociation confirmed the binding of PR1 to HLA-A2.1.

Induction of Primary CTL responses to peptides. These peptides were next used to stimulate T cells specific for peptide-coated targets. PBMC from a normal healthy donor heterozygous for HL-A2.1 were stimulated with peptide-pulsed T2 cells. The T2 cell line has been used by others as an antigen presenting cell for the generation of peptide-specific CTL. Briefly, T2 cells (which co-express the costimulatory molecule B7.1) were washed 3 times in serum-free RPMI culture media supplemented with penicillin/streptomycin and glutamine (CM) and incubated with peptide at 20 μg/mL for 2 hr in CM. The peptide loaded T2 cells were then irradiated with 7500 cGy, washed once, and suspended with freshly isolated PBMC at a 1:1 ratio in CM supplemented with 10% human serum (HS) (Sigma, St. Louis, Mo.). After 7 days in culture, a second stimulation was performed and the following day, 60 IU/ml of recombinant human interleukin-2 (rhIL2) (Biosource International, Camarillo, Calif.) was added. After 14 days in culture a third stimulation was performed, followed on day 15 by addition of rhIL-2. A fourth stimulation was performed on day 21 followed on day 22 by the addition of rhIL2. After a total of 27 days in culture, the peptide-stimulated T cells were harvested and tested for peptide-specific cytotoxicity toward Calcein AM-labeled T2 cells, leukemia cell lines, and fresh human leukemia cells, as merely exemplary embodiments. Such studies are extrapolated to breast cancer cells using similar procedures.

The CTL line generated against the PR1 peptide demonstrated high specific lysis against PR1-loaded target cells. CTL response toward PR1 was shown to be specific for target cells expressing the HLA-A2.1 molecule. This and the cytotoxicity observed was HLA-A2.1-restricted.

PR1-specific CTL preferentially lyse human myeloid leukemia cells, as exemplary embodiments. It was next determined whether the PR1-specific CTL line was capable of lysing allogeneic human myeloid leukemia cells from HLA-A2.1 positive individuals. As controls, two cell lines expressing low levels of Pr3 were used: HLA-A2.1 transfected K562 cells and U937 cell line which lack HLA-A2.1 and would therefore be incapable of presenting peptides in an HLA-A2.1-restricted manner. Cryopreserved bone marrow cells from patients P1-P4, and marrow cells from a healthy normal volunteer (D2, a bone marrow donor for an allogeneic bone marrow transplant performed on patient P3) were thawed and used as targets for the PR1-specific CTL line. There was specific lysis by PR1-specific CTL, at various E:T ratios, of either U937 cells, HLA-A2.1-transfected K562 cells, or T2 cells with or without exogenously added PR1 peptide at 1.0 μg/ml.

### Identifying Potential HLA-A2.1-Restricted Peptide Epitopes in PR3 to Elicit Leukemia-Reactive Human CTL Using Immunological Methods

To determine whether CTL reactivity to the PR1 peptide can be enhanced by single amino acid substitutions in the HLA-A2.1 anchor motif positions. As previously stated, it is known that certain amino acid substitutions in a peptide may enhance binding of the peptide to HLA-A2.1, which may subsequently enhance target recognition by CTL (Rosenberg et al., 1998). Thus, homologous PR1 peptides that contain single or double amino acid substitutions at the HLA-A2.1 residues, for example anchor residues, will be used to coat T2 cells and test for specific lysis. Some peptides may have higher binding affinities to HLA-A2.1 based on the same algorithm used to predict the PR1 peptide (Parker et al., 1994). These peptides will be synthesized (Biosynthesis Co.), tested for HLA-A2.1 binding using T2 cells, and tested in the mini-cytotoxicity assay where specific lysis will be compared to native PR1-coated T2 cells using TCL that are PR1-specific. The PR1-specific bulk culture CTL will be generated using the methods described herein.

In some embodiments, combinations of PR1 with PR1-derived peptides will be used to coat T2 target cells to test for specific lysis. Using CTL specific for this peptide, T2 cells will be coated with a fixed concentration of the peptide at 10 μg/mL, plus serial dilutions of PR1 (0.1 to 50 μg/mL) to test for potential interference with TCR recognition, as measured by reduced specific lysis at fixed E:T ratios. The results of these studies plus the IC₅₀ of each of the peptides will be used to make comparisons of which are the possible dominant and subdominant peptides. This will be used to develop vaccines using combinations of peptides to stimulate CTL immunity (Nestle et al., 1998).

If peptides used to coat T2 cells do not result in greater cytotoxicity over PR1-coated T2 cell targets, then PR1-specific CTL that are obtained after PR1-tetramer sorting will be studied for their ability to recognize and lyse the PR1-variant peptide-coated T2 cells. Because these cells are a much more homogeneous population of CTL, they are expected be a more sensitive indicator of improved CTL immunity.

### Antigen-Presenting Cells (APC) Elicit Immunity Directed Against CML

PR1-specific CTL with high and low TCR avidity can be elicited from healthy donors. To determine whether high and low avidity PR1-CTL are present in healthy donors, a modified tetramer staining technique (Savage et al., 1999) using limiting tetramer concentration to visualize high and low fluorescence intensity tetramer+ cells, which correlates with high and low TCR avidity was utilized. Sufficient PR1-CTL was elicited by stimulating PBMC with increasing peptide concentrations for 28 days. PR1-CTL elicited with low (0.2 μM) PR1 showed a 3-fold longer t_{1/2} than PR1-CTL elicited with high (20 μM) PR1 (58 vs 19 min), which correlates with overall high and low tetramer fluorescence, respectively, and validates the use of overall tetramer fluorescence intensity to indicate relative TCR avidity.

CML target cell killing by PR1-specific CTL correlates with TCR avidity. To evaluate whether CTL lines with different TCR avidities showed differences in effector function, 4-week old PR1-CTL lines derived from a healthy donor or from patients with CML were tested for the ability to kill HLA-A2+ CML target cells from a patient with blast crisis CML, autologous chronic phase CML cells from patient CML #3 at time of diagnosis, or cells from healthy donors. High avidity PR1-CTL elicited with 0.2 μM PR1 showed nearly 2-fold greater lysis of the same CML BM cells on a per cell basis than did the low avidity PR1-CTL elicited with 20 μM PR1. The specific lysis of autologous BM cells by a PR1-CTL line derived from a CML patient (CML #3) using 0.2 μM PR1 was similar to the amount of lysis of CML BM cells by the low avidity PR1-CTL line derived from the healthy donor. Similarly, CTL from patients CML #1 and CML #2 elicited with 0.2 μM PR1 showed lysis of CML #3 BM cells of 24%±5% and 33%±6%, respectively, at an E:T ratio of 20:1.

High avidity PR1-specific CTL are present only in interferon sensitive CML patients in cytogenetic remission. It was previously shown that detection of functional PR1-CTL in CML patients correlates with a cytogenetic response to interferon-α (Molldrem et al., 2000). This indicated that high avidity PR1-CTL would only be present in IFN-sensitive patients. To address this possibility PBMCs from untreated HLA-A2+ patients with either blast crisis (CML #1) or chronic phase CML (CML #2) or a patient with chronic phase treated with IFN-α for 3 months (CML #3) were stimulated weekly with PR1. Only low-avidity PR1-CTLs could be elicited from any of the three patients. There were no detectable PR1-CTLs by tetramer staining in PBMCs prior to repeated peptide stimulation.

To further address the possibility that high avidity PR1-CTL would only be present in IFN-sensitive patients, two CML patients in a cytogenetic remission after 9 months of interferon treatment (CML #5 and CML #6 with 0% and 85% Ph+ cells, respectively), interferon resistant patients with no cytogenetic remission (CML #7 and CML #8), and one untreated newly diagnosed patient (CML #9) for the presence of PR1-CTL with high or low avidity TCR were studied. High avidity PR1-CTL were identified in both patients in a cytogenetic remission but in none of the interferon resistant or untreated patients. However, low avidity PR1-CTL could be identified in the interferon resistant patients, but totaled less than 0.1% of CD8+ cells. Furthermore, PBMC from untreated HLA-A2+ patients with either blast crisis (CML #1) or chronic phase CML (CML #2) or a patient with chronic phase treated with interferon-α for three months (CML #3) were stimulated weekly with PR1. Only low avidity PR1-CTL could be elicited from any of the three patients. This indicates that low numbers of high avidity PR1-CTL may be sufficient to contribute to cytogenetic remission in interferon sensitive patients, but leaves unanswered whether high numbers of low avidity PR1-CTL may contribute to remission.

High PR1 concentration and proteinase 3-overexpressing CML cells induce apoptosis of high avidity PR1-specific CTL. Previous studies showing that high affinity virus-specific T cells are eliminated by target cells infected with a high viral load (Alexander-Miller et al., 1998; Alexander-Miller et al., 1996a), and that CML cells frequently overexpressed proteinase 3 ((Molldrem et al., 1996; Molldrem et al., 1997), suggested that high avidity PR1-CTL might be undetectable in untreated CML patients due to selective elimination by CML cells that overexpress the target antigen. To demonstrate this, an equal number of PR1-CTL from a healthy donor were challenged with T2 cells pulsed with either high or low doses of PR1 and studied for evidence of apoptosis by Annexin V staining 16 to 18 hr later. High avidity PR1-CTL underwent apoptosis when challenged with high dose (20 μM) PR1 peptide, but not when challenged with low dose (0.2 μM) PR1. All high avidity PR1-CTL exposed to high dose PR1 were dead after 36 to 48 hr of co-culture. Apoptosis was abrogated in the presence of the BB7.2 blocking antibody to HLA-A2.1, and no apoptosis was observed when 20 μM of the irrelevant peptide Flu was used instead of PR1. In contrast, low avidity PR1-CTL did not undergo apoptosis when challenged with either high or low concentrations of PR1.

To determine whether CML cells similarly induced apoptosis of high avidity PR1-CTL, co-incubation studies were performed with HLA matched BM cells from CML patients followed by staining for Annexin V, for 16 to 18 hr after co-incubation. High avidity PR1-CTL underwent apoptosis by 18 hr after co-culture with BM from an HLA-matched patient with CML in chronic phase with 100% Ph+ cells. No apoptosis was induced by co-incubation with BM cells from an HLA-A2 negative CML patient with 100% Ph+ cells, or by co-incubation with BM cells from an HLA-A2+ healthy donor. In contrast, less than 1% of the low avidity PR1-CTL underwent apoptosis when challenged with either the HLA-A2+ or HLA-A2-CML cells. Similar overexpression of cytoplasmic proteinase 3 was observed in BM cells from each of the CML patients (2.8-fold higher in the HLA-A2+ cells and 3.3-fold higher in the HLA-A2-cells compared to healthy donor BM cells), and MHC I expression was similar in the two patient samples. Therefore, differences in apoptosis were likely due to differences in the amount of PR1 peptide presented on the CML cells.

High avidity PR1-CTL persist in IFN-sensitive CML patients off of all therapy. Since it has been shown that IFN-sensitive patients have high avidity PR1-CTL in peripheral blood that can kill CML, it was determined whether PR1-CTL maintain remission in patients in CR off therapy. Three patients in complete cytogenetic remission after discontinued IFN were studied. The patients had CML from 5 to 9 years, and were off IFN from 18 to 26 months prior to study. All patients continued to have bcr-abl transcripts by RT-PCR. Both high and low affinity PR1-CTL were identified in all patients, although only 25% to 30% of all PR1-CTL were of high affinity. Importantly, all of the high affinity PR1-CTL were functionally active since stimulation with either PR1 peptide or SEB induced γ-IFN production by CFC and upregulated CD69, whereas the low affinity PR1-CTL did not produce γ-IFN, but did upregulate CD69 (not shown). Notably, the high affinity PR1-CTL from UPN3 upregulated CD69 but did not produce γ-IFN when tested at 21 months, indicating a loss of anti-leukemia immune function 5 months prior to cytogenetic relapse and the simultaneous disappearance of PR1-CTL.

The only high affinity PR1-CTL were CD45RA+/CD28+/CCR7+/CCR5−, indicating an effector memory or possibly a naïve phenotype. In addition, the high affinity PR1-CTL had significantly higher expression of CD28 (p=0.03) and lower expression of CCR5 (p=0.01) than low affinity PR1-CTL, which suggested that low affinity PR1-CTL are terminally differentiated memory cells with little anti-leukemia activity. These results indicated that IFN treatment induces a long-lasting renewing population of high affinity PR1-CTL that continue to maintain lasting cytogenetic remissions in some patients after discontinuing IFN therapy. Loss of functional activity amongst high affinity PR1-CTL or the presence of only low affinity PR1-CTL suggests acquired tolerance, leading to eventual relapse. This early nonresponsiveness may reflect an anergic state, but loss of the PR1-CTL at the time of relapse may be due to deletion.

PR1 peptide vaccine can elicit PR1-CTL immunity in patients with myeloid leukemia. Preliminary data suggested that in myeloid leukemia patients in whom a PR1-specific CTL immune response could be elicited or increased, PR1-CTL would convey an anti-leukemia immune response and contribute to remission. To test this in refractory leukemia patients, a phase I/II vaccine study was initiated. HLA-A2+ patients with CML (interferon-resistant or relapsed after BMT), AML (smoldering relapse or ≧2nd CR) or MDS (RAEB or RAEBt) with no detectable antibodies to proteinase 3 (no detectable cANCA) were eligible. Patients that relapsed after BMT or those ineligible for BMT were also eligible for study. Pregnant patients, HIV+ patients, and those with known vasculitis were excluded. Patients were required to have immunosuppression (i.e. cyclosporine, steroids) discontinued 4 weeks prior to study entry.

Primary endpoints were (1) toxicity assessment including the induction of autoimmunity resembling Wegener's granulomatosis, the systemic vasculitis associated with cANCA antibodies; and (2) induction of an immune response assessed by cytokine flow cytometry (CFC) of γ-IFN and PR1/HLA-A2 tetramer staining of PBMC before and 3 weeks after the last vaccination. Secondarily, clinical responses were assessed by standard criteria with bone marrow biopsy, cytogenetic studies (standard chromosome banding) and molecular studies (PCR for bcr-abl or other known abnormalities such as t(15;17), inv16, etc.) 3 weeks after the last vaccination. Patients were seen and evaluated in clinic every 3 weeks during the study period. Both vialed PR1 peptide (NSC698102) and the adjuvant (Montanide ISA-51, NSC675756) were used in this study.

The overall study was divided into two parts: Phase I consisted of nine patients treated in cohorts of three at 1 of 3 dose levels of 0.25 mg, 0.5 mg, or 1.0 mg of PR1 peptide given subcutaneously in incomplete Freund's adjuvant (Montanide ISA-51) and GM-CSF 75 mg subcutaneously every 3 weeks for 3 injections. The Phase II part of the study enrolled patients in cohorts of 4 randomized to one of the same three PR1 peptide doses since none of the doses were eliminated on the basis of toxicity during the Phase I part of the study. A continuous reassessment model was used in the statistical design to assess best dose level using criteria of immune response (γ 2-fold increase in the number of PR1-specific CTL during the vaccine study period) and grade 3 or 4 organ toxicity. If any patient developed vasculitis and/or cANCA, the trial would be stopped, and if any individual patient developed grade 3 or 4 organ toxicity the vaccine would be withheld for that patient. Any dose level would be discontinued if the number of patients with grade 3 or 4 toxicity, divided by the number of patients evaluated for toxicity, is greater than or equal to ¾, 4/8, 5/12 or 6/16. Any dose level would be terminated if none of the first 12 patients in that dose level have an immune response. If an immune response was noted, patients would continue to be entered onto that dose level, by continued randomization, to a maximum of 20 patients per dose level. If any clinical response was noted during the study period either with or without a measurable immune response, this would be considered an efficacy endpoint and patients would be entered onto that dose level to a maximum of 20 patients per dose level. Patients were monitored every 3 weeks with chest x-rays, ANCA titers and physical examinations and for immune responses using PR1/HLA-A2 tetramers. Bone marrow cells (BMC) were obtained before the first vaccination and 3 weeks after the last vaccination to assess disease status.

### Example 4

### Exemplary Myeloperoxidase (MPO) Embodiments

Another myeloid-restricted protein, Myeloperoxidase (MPO), is a heme protein synthesized during early myeloid differentiation that constitutes the major component of neutrophil azurophilic granules. The present example provides exemplary but illustrative embodiments for peptides from MPO that are employed for leukemia but may be extrapolated to breast cancer embodiments also.

It was found that MY4 (RLFEQVMRI (SEQ ID NO:6)), a 9 aa peptide derived from MPO that binds to HLA-A2.1, can be used to elicit CTL from HLA-A2.1+ normal donors in vitro (Braunschweig et al., 2000). These MY4-specific CTL show preferential cytotoxicity toward allogeneic HLA-A2.1+ myeloid leukemia cells over HLA-identical normal donor marrow (Braunschweig et al., 2000). MY4-specific CTL also inhibit colony forming unit granulocyte-macrophage (CFU-GM) from the marrow of CML patients, but not CFU-GM from normal HLA-matched donors Like PR1, MY4 is therefore a peptide antigen that can elicit specific CTL lysis of fresh human myeloid leukemia cells. Other peptides from MPO are predicted to bind to HLA-A2.1, but not all of these have been tested for their potential to stimulate immunity.

Some previous studies have established PR1 to be an important leukemia-associated antigen (LAA), and because of the many striking similarities of the nature of the immunity directed against Pr3 and MPO, it is likely that similar methods applied to the study of MPO-specific immunity will establish MY4 and potentially other MPO peptides as important LAA as well (Kochenderfer and Molldrem, 2001).

### Peptide Selection and Binding Assays

In the first step to generating T cells which could be used for adoptive immunotherapy of myeloid leukemias, several peptides derived from the published sequence of MPO have been identified which were predicted to bind to HLA-A*0201 using a published algorithm (Molldrem et al., 1996; Parker et al., 1994). This allele was chosen because its high frequency in the US population (49% of individuals) would maximize the therapeutic relevance of any eventual immunotherapeutic strategy. Of 10 peptides predicted to have sufficiently high binding affinities based on the known HLAA2.1 binding motif, the five with the highest predicted binding were subsequently synthesized (designated MY1 through MY5) (Table 3). The peptides were synthesized by Biosynthesis (Lewisville, Tex.) or by the M. D. Anderson Protein CORE Facility (Houston, Tex.) to a minimum of 95% purity as measured by high-performance liquid chromatography (HPLC). Peptide binding to HLA-A2.1 was confirmed using two assays. In the first, indirect flow cytometry was used to measure HLA-A2.1 surface expression on the A2+ T2 cell line coated with the peptide. T2 cells are a human lymphocyte line that lacks TAP1 and 2 genes and cannot therefore present endogenous MHC class I restricted antigens. If the peptide effectively bound HLA-A2.1, it stabilized the complex with β2-microglobulin and increased HLA-A2.1 surface expression, which could be measured using flow cytometry. An HLA-A2.1 specific monoclonal antibody (BB7.2, ATCC, Rockville, Md.) followed by a FITC-labeled secondary antibody (CALTAG) was used to measure surface expression of HLA-A2.1. In the second assay, the dissociation rate of I¹²⁵-labeled β2-microglobulin from the heterotrimer complex of the HLA-A2.1 heavy chain, peptide, and β2-microglobulin was measured, which allowed calculation of binding half-life (t_{1/2}). The labeled heterotrimer complex was separated from unincorporated β2-microglobulin by high-performance liquid

TABLE 3
Peptide Start Amino Acid (aa) Subsequence
Residue Half-Time Position Disassociation

| | | | SEQ | Residue Half-Time |
|---|---|---|---|---|
| | Start | Subsequence | ID | Position |
| Peptide | (aa) | Residue | NO: | Disassociation |
| MY1 | 132 | SLWRRPFNV | 7 | 3348.233 |
| MY2 | 28 | KLLLALAGL | 8 | 636.279 |
| MY3 | 98 | LLSYFKQPV | 9 | 449.306 |
| MY4 | 571 | RLFEQVMRI | 6 | 364.011 |
| MY5 | 504 | LIQPFMFRL | 10 | 253.129 |
| MY6 | 529 | RVFFASWRV | 11 | 168.881 |
| MY7 | 53 | VLGEVDTSL | 12 | 148.896 |
| MY8 | 418 | LLLREHNRL | 13 | 134.369 |
| MY9 | 143 | VLTPAQLNV | 14 | 118.238 |
| MY10 | 118 | YLHVALDLL | 15 | 110.747 |

MPO peptides predicted to bind HLA-A2.1 chromatography gel filtration, and the halftime of disassociation of β2-microglobulin were determined by subjecting aliquots of the complex to a second round of gel filtration. Five peptides showed increased surface HLA-A2.1 expression compared with T2 cells with no added peptide (background HLA-A2.1 expression; (Braunschweig et al., 2000). The control peptides are the PR1 peptide and an Influenza B nucleoprotein (aa 85-94; Flu), both with known high binding affinity to HLA-A2.1. The long measured t1/2 as measured using β2-microglobulin disassociation confirmed the binding of MY1 through MY5 to HLAA2.1 (Molldrem et al., 1996).

### Induction of Primary CTL Responses to Peptides

The five MPO peptides discussed above were used to stimulate T cells specific for peptide-coated targets. PBMC from a normal healthy donor heterozygous for HLA-A2.1 were stimulated with peptide-pulsed T2 cells. The T2 cell line has been used by others as an antigen presenting cell for the generation of peptide-specific CTL. Briefly, T2 cells (which co-express the costimulatory molecule B7.1) were washed 3 times in serum-free RPMI culture media supplemented with penicillin/streptomycin and glutamine (CM) and incubated with peptide at concentrations ranging from 0.2 to 200 μg/mL for 2 hr in CM. The peptide loaded T2 cells were then irradiated with 7500 cGy, washed once, and suspended with freshly isolated PBMC at a 1:1 ratio in CM supplemented with 10% human serum (HS) (Sigma, St. Louis, Mo.). After 7 days in culture, a second stimulation was performed and the following day, 60 IU/mL of recombinant human interleukin-2 (rhIL-2) (Biosource International, Camarillo, Calif.) was added. After 14 days in culture a third stimulation was performed, followed on day 15 by addition of rhIL-2. A fourth stimulation was performed on day 21 followed on day 22 by the addition of rhIL-2. After a total of 27 days in culture, the peptide-stimulated T cells were harvested and tested for peptide specific cytotoxicity toward CalceinAM labeled T2 cells, leukemia cell lines, and fresh human leukemia cells. No peptide specific CTL lines could be elicited using MY1, MY3 or MY5 peptides, despite testing using different donors and differing peptide concentrations. The CTL line generated against the MY2 peptide demonstrated high specific lysis against MY2-loaded target cells, whereas the CTL line generated against MY4 did not demonstrate any significant cytotoxicity against MY2-loaded targets (Molldrem et al., 1996). The converse experiment, using CTL generated against MY4, tested similarly. Cytotoxicity toward T2 cells loaded with HTLV-1 tax (aa 11-19), an irrelevant peptide with high binding affinity to HLAA2.1, was also measured and resulted in <20% specific lysis at E:T ratios of 50:1 by CTL specific for either MY2 or MY4. CTL stimulated weekly with either higher or lower peptide concentrations of MY2 or MY4 did not produce a short-term CTL line by 4 to 6 wk, as measured by specific lysis of peptide-coated T2 targets at the end of culture. Only CTL stimulated with 2.0 μg/ml of peptide produced effective short-term CTL lines. This observation was reproducible across 10 healthy HLA-A2.1+ donor PBMCs that were used in the studies to elicit the CTL. This phenomenon was noted previously for CTL elicited against other self-peptides.

### HLA-A2.1 Restricted CTL Responses

To further demonstrate that the CTL response toward MY2 or MY4 are specific for target cells expressing the HLA-A2.1 molecule, T2 cells loaded or not loaded with 2.0 μg/mL MY2 or MY4 were prepared. The CTL line generated against the respective peptides were also used to test for specific lysis. Mouse monoclonal antibody against HLA-A2.1 (BB7.2) was used to block HLA-A2.1-restricted recognition by the CTL line. T2 cells without peptide, but with antibody present, were used to control for any potential non-specific antibody-mediated cytotoxicity.

With the addition of antibody to HLA-A2.1, specific lysis was blocked. Further, there was only background lysis of T2 cells in the presence of antibody alone. This demonstrated that the observed cytotoxicity was HLA-A2.1-restricted.

### Specific CTL Preferentially Lyse Human Myeloid Leukemia Cells

It was next determined whether the MY2 and MY4-specific CTL lines were capable of lysing allogeneic human myeloid leukemia cells from HLA-A2.1 positive individuals. The targets were BM cells from pre-transplant HLA-A2.1-positive AML patients. As controls, BM cells from HLA-A2.1-negative AML patients, BM from HLA-A2.1-positive healthy donors and two cell lines expressing low levels of MPO were used: HLA-A2.1 transfected K562 cells and the U937 cell line which lacks HLA-A2.1 and would therefore be incapable of presenting peptides in an HLA-A2.1-restricted manner.

The specific lysis by MY4-specific CTL, at various E:T ratios, of either BM from healthy HLA-A2.1-positive donors, HLA-A2.1-positive AML patients, HLA-A2.1-negative AML patients, or T2 cells with or without exogenously added MY4 peptide at 2.0 μg/mL is shown. The specific lysis of U937 and HLA-A2.1-positive K562 cells by MY4-specific CTL was lower than the background lysis observed against T2 cells without added peptide. The results show that CTL elicited with the MY4 peptide result in short-term CTL lines with both MY4 and HLA-A2.1 specificity that killed AML cells, but not normal cells.

In contrast, cytotoxicity results from three experiments with three different MY2-specific CTL lines demonstrate that marrow cells from patients with HLA-A2.1-positive AML were readily lysed, at an E:T ratio of only 5:1. However, marrow cells taken from an HLA-A2.1-positive normal healthy donor also demonstrated significant lysis (43% lysis at E:T of 20:1), similar to that of the HLA-A2.1-positive AML cells.

MY2- and MY4-Specific CTL Lysis of Leukemia Cells is Associated with Aberrant MPO Expression

All target cells were assayed for the presence of cytoplasmic MPO. After permeabilizing the cell membrane with Ortho PermeaFix (Ortho Diagnostics, Raritan, N.J.), staining was performed using a FITC-labeled antibody to MPO (Accurate Chemicals, Westbury, N.Y.) and a PE-labeled antibody to CD34 (Becton-Dickinson, San Jose, Calif.) followed by flow cytometry.

The percentage of cells expressing surface MHC class I and CD80 (the costimulatory molecule B7.1) was also evaluated in the same target cell populations by staining with FITC labeled antibodies. These experiments demonstrate that it is possible to elicit CTL specific for the MY2 and MY4 self-peptides from normal HLA-A2.1-positive donors that exhibit in vitro cytotoxicity against myeloid leukemia cells. Furthermore, the degree of cytotoxicity was associated with aberrant MPO expression. However, the MY2-specific CTL also showed specific lysis of normal donor marrow cells, which suggests that immunity elicited against this peptide in vivo might result in autoimmunity that would be incapable of distinguishing leukemic cells from normal marrow progenitor cells. Lastly, since the CTL were tested against whole marrow from leukemia patients in short-term assays, it was possible that leukemia progenitor cells, which might not aberrantly express MPO, could escape CTL recognition. Therefore, whether leukemia progenitor cells could be eliminated by MY4-specific CTL in an AML colony-forming assay was investigated. The MPO expression in both leukemia and normal CD34+ cells was also determined.

### MY4-Specific CTL Preferentially Inhibit AML Colony-Forming Units

PBMC from two normal healthy donors heterozygous for HLA-A2.1 were stimulated with peptide-pulsed T2 cells using the method previously described. In colony inhibition assays using CTL derived from a 13 day MY4 peptide-pulsed culture (CTL1). CFU-GM from patient P1 (M2-AML) showed 63% (p=0.006) inhibition. In contrast there was no inhibition of CFUGM from normal marrow, D1 and D2, the corresponding HLA identical marrow donors for P1 and P2. Control CTL1 plated alone in methylcellulose under identical experimental conditions at 5×10⁵ cells/ml showed no CFU-GM by day 16.

### MPO is Expressed in Leukemic CD34+ Cells-Expression is Limited to Hematopoietic Cells

Next it was confirmed that MPO was expressed in early CD34 positive CML cells. Marrow was obtained from a patient with CML in CP, a patient with AML, and normal CD34 cells from G-CSF mobilized peripheral blood mononuclear cells from a normal donor for comparison. Cells were first labeled with PE conjugated anti-CD34 antibody (Becton Dickinson, San Jose, Calif.), followed by cytoplasmic staining for MPO.

Nineteen percent of the CML cells were CD34 positive and 16% of those cells highly expressed Pr3. In addition, CD34-negative cells also expressed MPO. In contrast, none of the normal CD34 positive cells expressed MPO. In cells from another patient with AML, 57% of AML cells were CD34-positive and 5% of those cells highly expressed MPO. This shows that very early progenitor cells overexpress MPO whereas there is no MPO expression normal progenitor cells.

To confirm that MPO expression was limited to hematopoietic cells, a panel of human tissues for MPO RNA expression was analyzed using RT-PCR. Expression of MPO is limited to bone marrow.

### MY4-Specific CTL Identified in Peripheral Blood of Recipients of Nonmyeloablative Stem Cell Transplants Using Peptide-HLA-A2 Tetramers

Because CTL lines against both MY2 and MY4 could be elicited from normal donors and that killed AML, it was next assessed whether it was possible to detect these CTL in the peripheral blood of patients with AML. In contrast to CTL with specificity for MY4, MY2-specific CTL caused lysis of both leukemic and healthy bone marrow cells, which suggested that it would be unlikely to find high circulating numbers of MY2-specific CTL since these might mediate autoimmunity in addition to anti-leukemia immunity. Previous successful methodology using peptide/HLAA2 tetramers to identify the similarly deduced peptide PR1 as an HLA-A2.1-restricted leukemia associated antigen (LAA) (Molldrem et al., 2000) argued strongly that this same methodology could be used to determine whether MY2 and MY4 were also potential LAAs.

Production of peptide/MHC tetramers has been described by in detail elsewhere (Molldrem et al., 2000). Briefly, a 15 amino acid substrate peptide (BSP) for BirA dependent biotinylation has been engineered onto the COOH terminus of HLA A2. The A2 BSP fusion protein and human β2 microglobulin (β₂M) were expressed in E. coli, and were folded in vitro with the specific peptide ligand. The properly folded MHC peptide complexes were extensively purified using FPLC and anion exchange, and biotinylated on a single lysine within the BSP using the BirA enzyme (Avidity, Denver, Colo.). Tetramers were produced by mixing the biotinylated MHC peptide complexes with phycoerythrin (PE) conjugated Neutravidin (Molecular Probes), or PE(Cy7)-conjugated Neutravidin at a molar ratio of 4:1. MY2 and MY4 tetramers were validated by staining against a CTL line specific for each peptide. CMV tetramers were validated by staining with PBMC from a CMV immune individual. Specificity was demonstrated by the lack of staining of irrelevant CTL. By titrating positive CTLs into PBMCs from normal controls, the limit of detection was established to be as low as 0.01% of CD8+ cells. Each tetramer reagent was titered individually and used at the optimum concentration, generally 20 μg/ml-50 μg/ml.

Nine HLA-A2.1-positive AML patients in relapse prior to allogeneic NST, and then again at day 60 post-transplant were examined using peptide/MHC tetramers. All of the patients were CMV immune prior to transplant, and therefore pp65/HLA-A2.1 tetramers served as positive controls for evidence of antigen specific immunity. Because of the limited amount of sample that was available, staining method was modified to use 2 or more different tetramers simultaneously to stain PBMC samples.

In contrast to MY2-CTL, which showed 45% specific lysis of HLA-A2+ normal BMC in addition to killing leukemia, MY4-CTL showed no lysis of normal BMC and only killed leukemia cells, which suggested that MY4 may be a more biologically relevant leukemia antigen. It was predicted that high numbers of circulating MY2-CTL would not be found, but that MY4-CTL may be detectable in leukemia patients. CTL from blood samples obtained 60 days after NST in 9 HLA-A2 AML patients were studied using combinations of 6 HLA-A2 tetramers and multiparameter flow cytometry using a MoFlo cytometer (Cytomation, Fort Collins, Colo.). A2 tetramers were constructed with the following peptides: PR1, MY2, MY4, the CMV pp65 peptide, and the minor antigens HA-1R (a negative control) (den Haan et al., 1998) and HA-1H (the allele against which CTL responses have been shown) (den Haan et al., 1995; den Haan et al., 1998; Marijt et al., 1995). All patients showed evidence of donor chimerism by DNA microsatellite analysis at the time of study, and all were CMV seropositive.

Multiple-tetramer staining is associated with a 0.1% loss of sensitivity; however, ratios of antigen specific CTL were preserved when compared to single tetramer staining. Staining PBMC from HLA-A2.1-positive healthy donors and from patients with lymphoid-derived tumors (multiple myeloma and chronic lymphocytic leukemia) showed there was no detectable CTL immunity against myeloid-specific antigens. However, both PR1-CTL and MY4-CTL are detected in the peripheral blood of these patients, but MY2-CTL is not detected (the limit of sensitivity is 0.01% in cell titration experiments).

Next, a total of 9 HLA-A2+ NST recipients were examined on day 60 post-transplant for immunity against each of the 6 peptides. By comparing relapse rates, a greater percentage of PR1-CTL were present in patients in continuous remission at 1 year (median of 2.45%) versus those patients that went on to relapse (median of 1.55%), p=0.03. Although there was a trend toward a higher percentage of both MY4- and HA-1H-CTL in patients in remission, it did not reach statistical significance. No MY2-CTL were identified in any of the patients studied. The development of either >grade II acute GvHD or chronic GvHD did not correlate with the percentage of any of the peptide-specific CTL studied, although there was a trend toward a higher number of HA-1-CTL in patients that developed significant GvHD. Importantly, these preliminary experiments (1) extended the importance of PR1-specific CTL immunity to NST recipients, (2) highlighted the probable relevance of MY4-specific CTL immunity in NST recipients, and (3) lessened the likelihood of biological relevance of MY2-specific CTL immunity as part of the anti-leukemia response.

This is the first study to combine simultaneous multi-tetramer analysis with other surface phenotypic markers to determine the relative importance of leukemia-associated antigens in the GVL effect. These results indicate that although CTL with MY2 specificity are below the detection limit using tetramer staining, MY4-CTL and HA-1H-CTL are increased in NST recipients and may therefore be important in the GVL effect after NST for AML. These results also extend the previous results and suggest that PR1-CTL may also contribute to the elimination of AML after NST.

### Sorted Antigen-Specific CTL Specifically Kill Leukemia Cells

To show that tetramer-sorted antigen-specific CTL are functional, PR1/HLA-A2 tetramer+ CTL from a donor lymphocyte (DLI) product obtained from leukapheresis were stained, sorted and tested for lysis of both donor and recipient (which contained >90% blasts) cryopreserved BM. The recipient was in remission by 6 months after allogeneic BMT, but relapsed with chronic phase CML by 12 months with 100% Ph+ BM cells. The patient was then treated with a total of 7×10⁷ DLI per kilogram body weight from months 12 to 13 with no other therapy, and was in remission with 0% Ph+ BM cells by month 18 when PBMC were available for testing.

After staining with the PR1/HLA-A2 tetramer and sorting on the MoFlo cytometer, the yield of sorted PR1/HLA-A2 tetramer+ cells was 81%, with 90% purity, and the sorted tetramer-negative population contained no detectable PR1/HLA-A2 tetramer+ CTL. The sorted PR1/HLA-A2 tetramer positive CTL showed greater lysis of recipient marrow taken from time of relapse than the non-sorted PBMC. Although the sorted PR1/HLA-A2 tetramer negative CTL showed less lysis of recipient BM than non-sorted PBMC, it was above background lysis against donor BM. This likely reflects non-PR1-specific CTL with activity against other leukemia antigens. Minimal or no lysis of donor BM was seen with either the sorted PR1/HLA-A2 tetramer positive CTL or non-sorted PBMC. This is strong evidence that PR1-specific CTL actively lyse CML cells and contributed to remission in this patient (Molldrem et al., 2000).

To confirm leukemia specificity of the PR1-specific CTL, PBMC from the recipient were again sorted using the PR1/HLA-A2 tetramer and tested for lysis of HLA mismatched target cells. PR1/HLA-A2 tetramer sorted PBMC showed lysis of HLA-A2.1+ CML cells from 2 unrelated patients, but no lysis of either HLA-A2.1-CML cells or HLA-A2.1+ normal donor marrow cells at E:T ratio of 5:1.

To show that CTL with specificity for peptides such as MY4, which are identified using a deductive strategy, can successfully be translated to the clinic, a phase I clinical trial using the PR1 peptide as a vaccine with incomplete Freund's adjuvant and GM-CSF was initiated. This further demonstrates that highly useful LAA can be identified using these methods.

Patients eligible for the vaccine included HLA-A2+ CML and AML patients that had failed conventional therapy or AML patients that were in 2nd CR (i.e., at high risk of relapse). When PR1 was administered subcutaneously at 0.25, 0.5 or 1.0 mg every 3 weeks for 3 injections, PR1-specific CTL immunity was elicited in 6 of 9 patients (by tetramer staining) and complete remissions were obtained in 2 patients (1 AML and 1 CML patient). The patient with AML was positive for the t(15;17) translocation and subsequently became PCR negative after vaccination. Importantly, the expanded PR1-specific CTL from peripheral blood of that patient were isolated by tetramer staining and relapsed BM cells were killed, but not BM cells taken during remission. This technique may be applied to the treatment of other forms of leukemia, to other HLA types and potentially to other tumors as well.

Thus, to develop effective leukemia-specific immunotherapies, Pr3 and MPO were investigated as tissue-restricted proteins and it was found that the HLA-A2.1-restricted self-peptides, PR1 and MY4, derived from Pr3 and MPO, respectively, can be used to elicit peptide-specific CTL that preferentially attack myeloid leukemia based on aberrant expression of the parent proteins in the target cells. By using tetramers to study post-transplant and postinterferon treated patients for peptide-specific immunity, PR1 was established as a leukemia-associated antigen.

The data indicate that PR1 and MY4 could be used as target antigens to stimulate both active and passive leukemia-specific immunity. MY4-specific CTL will be given in an adoptive immunotherapy study with nonmyeloablative stem cell transplant, and in a clinical phase I trial other peptide antigens that are identified will be added to this approach. MY4-specific CTL will be selected and expanded ex vivo with the MY4 antigen for the production of leukemia-reactive CTL to produce a GVL effect and minimize GVHD.

A deductive strategy to identify peptide antigens from myeloperoxidase that are restricted to common HLA alleles that can be used to elicit leukemia-specific CTL responses in vitro. It has been shown, as previously discussed, in 9 HLA-A2+ AML patients who received NST that, in addition to PR1, MY4 is another new potential LAA. However, only 48% of the U.S. population has the HLA-A2 allele. Therefore, to extend any resulting immunotherapy strategies based on newly identified peptide antigens, whether each of the peptides predicted to have high binding (dissociation half-time >10) to the HLA-A3 and the HLA-B7 alleles can also elicit peptide-specific CTL will be addressed.

TABLE 4
Exemplary MPO Peptides

| Starting | | |
|---|---|---|
| Amino Acid | | SEQ ID |
| Position | Sequence | NO |
| 466 | VLGPTAMRK | 16 |
| 595 | GLPGYNAWR | 17 |
| 503 | TLIQPFMFR | 18 |
| 571 | RLFEQVMRI | 19 |
| 62 | VLSSMEEAK | 20 |
| 508 | FMFRLDNRY | 21 |
| 471 | AMRKYLPTY | 22 |
| 452 | AMVQIITYR | 23 |
| 663 | CIIGTQFRK | 24 |
| 361 | GLLAVNQRF | 25 |
| 434 | NPRWDGERL | 26 |
| 234 | IVRFPTDQL | 27 |
| 468 | GPTAMRKYL | 28 |
| 136 | RPFNVTDVL | 29 |
| 575 | QVMRIGLDL | 30 |
| 522 | NPRVPLSRV | 31 |
| 588 | MQRSRDHGL | 32 |
| 191 | SNRAFVRWL | 33 |
| 325 | TIRNQINAL | 34 |
| 352 | NLRNMSNQL | 35 |

Peptides of Table 4 may be employed in the invention. To determine whether the remaining peptides from Table 4 will also bind to HLA, the HLA-A3 and HLA-B7 alleles from EBV-transformed B cells derived from HLA-A3+ and HLA-B7+ normal donors were first cloned. These genes were inserted into the BirA-containing cassette that was used to construct the HLA-A2.1 tetramers and were then used to fold HLA-A3 and HLA-B7 tetramers using peptides with known high binding affinity to the respective alleles. Tetramers folded with the newly identified peptides will be used as reagents to test whether patients have evidence of circulating peptide-specific CTL.

The peptide-specific CTL lines generated in vitro from healthy donors that show peptide-specific lysis will be used as “reagents” to confirm the specificity of the tetramers. The A3 and B7 alleles have also been cloned into a mammalian vector containing the CMV promoter (Clonetech). Electroporation will be used to transduce T2 cells with these vectors. The transduced T2 cells will then be expanded for up to 1 month and sort-purified using the MoFlo high-speed cell sorter based on increased A3 or B7 surface expression after the addition of stabilizing A3- and B7-binding peptides.

The resulting T2 cells can then be used to determine whether the peptides from Table 4 bind to A3 and B7 by using A3- and B7-specific monoclonal antibodies (Immunotech and Pharmingen), and measuring surface expression. These binding results will be compared to peptides with known binding affinities to A3 and B7, such as influenza matrix and CMV pp65-derived peptides. The relative binding affinities of these peptides to the HLA allele will be determined by serial dilutions of each peptide and comparing them to PR1 after analyzing for surface HLA expression by flow cytometry. In this way, an IC50 value will be determined for each peptide.

Using the methods described for PR1 and the first 5 MPO peptides examined, the resulting peptide-elicited CTL lines will be characterized for their ability to kill peptide-coated T2 cells, fresh leukemia cells and established leukemia cell lines such as U937 and K562. HLA restriction will be confirmed using targets without the relevant allele and by blocking experiments with antibodies specific for the relevant alleles. The amount of target cell killing will be determined using a standard 4-hr assay (Molldrem et al., 1996; Hensel et al., 1999) and will be correlated with target antigen expression and surface phenotype of the leukemia cells and healthy donor BM cells.

MPO intracellular protein expression will be determined using direct intracellular FACS staining for the MPO protein with a FITC-labeled murine monoclonal antibody. This intracellular stain will be combined with surfaced antibodies for myeloid differentiation markers such as CD34, CD33, CD13, CD14, CD16 and HLA-DR to determine which stage of differentiation might be more susceptible to CTL killing. The MoFlo cytometer is capable of simultaneous 10-color analysis, which will greatly facilitate the analysis of progenitor stage of development. Both BM and PBMC will be examined similarly for MPO expression and surface phenotype and compared to determine whether there are differences in target susceptibility based on location (marrow vs. peripheral blood).

Determining whether CTL with specificity for myeloperoxidase-derived peptides can be detected in vivo in patients at diagnosis before and after NST and after treatment with chemotherapy. Because CTL lines against both MY2 and MY4 could be elicited from normal donors and kill AML cells, it was next determined whether it was possible to detect these CTL in the peripheral blood of patients with AML. In contrast to CTL with specificity for MY4, MY2-specific CTL caused lysis of both leukemic and healthy bone marrow cells, which suggested it would be unlikely to find high circulating numbers of MY2-specific CTL since these might mediate autoimmunity in addition to anti-leukemia immunity. Previous studies using peptide/HLA-A2 tetramers to identify the similarly deduced peptide PR1 as an HLAA2.1-restricted leukemia associated antigen (LAA) (Molldrem et al., 2000) argued strongly that the same methodology could be used to determine whether MY2 and MY4 were also potential LAAs. Thus, paired samples from 90 AML patients treated using nonmyeloablative transplant regimens (up to 30 per year for the first 3 years) will be examined. Patients will be studied for immunity to the 6 peptides discussed, in addition to any peptide antigens that are found to elicit anti-leukemia immunity. This will include peptides with HLA-A3 and HLA-B7 restrictions. Patient peripheral blood samples will be obtained prior to transplant and then weekly after transplant, beginning on day 10 and continuing until day 100. Patient samples will then be examined at each follow-up in the BMT clinic, which will be monthly until 1 year post-transplant. PBMC samples will also be obtained from the donor pre-transplant. BM cells will be obtained from the donor if BM is used as the graft, and from the recipient prior to transplant and again on days 30, 100 and day 365 post-transplant. The PBMC and BM samples will be cryopreserved. PBMC samples will be used for later evaluation as more peptides are identified as potential LAAs. The lymphocytes for surface expression of several markers, will be examined including CD3, CD4, CD8, CD16+56, CD45RA, CD45RO, CD57, CD28, CD27 as well as tetramer staining.

The maximum number of tetramer+ cells during the time course of study will be determined. Prior experience with viral antigen-specific CTL, with HA-1-specific CTL and with PR1-specific CTL suggest that the peak number of tetramer+ cells occurs over a 3 to 4 week period and often coincides (or may lag by a week or two) with the time of documented remission. Furthermore, the peak for other peptide-specific CTL is usually in the range of 1% to 10%. Significant increases over baseline of tetramer+ cells occurring during the study period will be verified. The study size will provide 85% power to establish a mean absolute increase of 1 percentage point (SD=3.5% based on previous data, type I error=0.05) at peak. In addition, the proportion of patients who show tetramer positivity, defined as at least 1% of cells positive at peak (SD+/−10% under assumed rate of 50% positivity) will be estimated. BM cells will be studied for MPO expression using intracytoplasmic staining combined with surface phenotypic makers that will allow the determination of the point of maturation of the BM cells. Expression of CD11a, CD13, CD14, CD16, CD33, CD34, CD80, CD86, HLA-ABC and HLA-DR will be examined. Because MY4 and the other peptides in this study are self-antigens, it is possible that AML patients treated with chemotherapy alone may have circulating numbers of MY4/MHC tetramer+ cells based on a previous study of CML patients using the PR1/HLA-A2 tetramer, however, this would seem unlikely. If these peptides are detected in AML patients that are in remission it may indicate that post-chemotherapy recovery of immunity is important for obtaining remission and the length of remission duration. Blood samples will be obtained from 10 consecutive AML patients receiving chemotherapy as alternative to NST, with samples obtained at the same time points after start of therapy as for the NST group. Thus, whether the proportion of patients showing tetramer positivity differs between the chemotherapy and NST groups will be addressed. It is anticipated that less than 5% of chemotherapy patients will show positivity compared to about 50% of NST patients. A chi-square test comparing the two proportions will have 92% power to detect the difference of interest (type I error=0.05), comparing the 10 chemotherapy patients to 90 NST patients. To determine the significance of circulating tetramer+ cells, it will also be assessed whether the presence of MY4 tetramer+ lymphocytes is associated with duration of disease response. All or nearly all of the 90 patients who start NST will be in complete remission or achieve it following NST therapy. From 30 to 40 relapses will have occurred at the time of data analysis, one year after the last patient is treated. Patients will be classified into two groups based on whether or not the patients have detectable tetramer+ cells. The association of response duration to tetramer positivity status will be modeled assuming proportional hazards. The study is powered to detect a tripling in risk of relapse associated with failure to detect tetramer+ lymphocytes. In addition, separate assessments of the association of tetramer positivity with duration of response determined by molecular and cytogenetic methods, will be made. It will also be important to determine whether the MY4-CTL are functional. Various methods of assessing function have been described, including cytokine secretion, CD69 upregulation, cell proliferation, and cytotoxicity. Tetramer staining and cytokine flow cytometry (CFC) will simultaneously be determined on all patients since the MoFlo will greatly facilitate these experiments. PE-labeled antibody to gamma-interferon and PECy7-labeled tetramers will be used to in these studies. Cells will first be labeled for 10 min at 37° C. with tetramer and FITC-labeled CD8 and then stimulated with MY4 peptide at 2 μg/ml. Brefeldin A will be added during culture to inhibit secretion of cytokines, and after 6 hr cells will be permeabilized and stained for interferon. This technique has been successfully used to monitor PR1-CTL responses after vaccination and it was found that CFC positivity correlates with cytotoxicity. In select patients with sufficient numbers of available PBMC and tetramer+ cells, the tetramer+population will be purified by high-speed sorting using the MoFlo cytometer. Both the tetramer+ and tetramer-cells will be tested for cytotoxicity against cryopreserved leukemia targets prior to NST or chemotherapy. Because the MoFlo is capable of 4-way simultaneous sorting, the killing of peptide-coated target cells of the MY4-CTL will be compared to other peptide-specific CTL (i.e. pp65 in serpositive patients) to directly compare lytic potential.

To determine the optimal method to select and expand peptide-specific CTL from healthy donors that can be used for adoptive cellular immunotherapy of NST recipients. Because CTL with myeloid self-antigen specificity are present at very low precursor frequencies (Molldrem et al., 1999), these CTL will need to be expanded before they could be used as part of an adoptive transfer immunotherapy strategy. While there are currently some commercially available methods for the separation of antigen-specific CTL based on cytokine secretion, they are presently not licensed for use in patients, and the best method to separate antigen-specific CTL has not been defined. Similarly, the optimal method to expand CTL in short-term culture conditions is not yet defined, and expansions on large scale without the use of fetal calf serum remain a major obstacle to cellular immunotherapy. Two general expansion methods to elicit MY4-CTL in vitro will be compared.

In the first approach, peptide-pulsed dendritic cells (DCs) will be used to stimulate responder PBMC weekly for 4 to 6 wk. Although there are many potential sources for precursors to mature DC, including CD34+ cell-derived hematopoietic precursors, monocyte-derived DC have been chosen because they are more readily available in large numbers from donor leukapheresis products. For practical reasons, this methodology is most likely to yield the greatest potential number of DC, which will be needed to grow low precursor frequency self-antigen specific CTL. For similar practical reasons, the use of interferon (IFN) and GM-CSF to grow DC, with or without TNF-α added to mature the cells during the last 48 to 72 hr of culture have been studied. The advantage is that both IFN and GMCSF are commercially available and have been used extensively in humans. In brief, DCs were grown using combinations of either 1,000 U/ml IL-4 plus 500 U/ml GM-CSF (termed DC/4GM) or 1,000 U/ml interferon-α2b plus 500 U/ml GM-CSF (termed DC/IGM). Previously cryopreserved PBMC from HLA-A2+ healthy donors were thawed, washed and adhered to plastic flasks prior to the addition of media +10% human serum (HS) with the addition of the above cytokines. T2 cells were maintained in RPMI+10% HS prior to co-culture with donor PBMC. At the end of 7 days, DC were pulsed with 20 μg/ml PR1 peptide, irradiated and combined with fresh PBMC from the same donor at a 1:2 ratio. On day 7, the culture was restimulated with PR1-pulsed DC (or T2) and on day 8 IL-2 at 20 U/ml was added to the cultures. Restimulation and IL-2 addition was repeated weekly until day 26 through 28 when the PR1-CTL cultures were tested for their ability to lyse PR1-coated target cells or CML cells. The PR1-CTL were also evaluated for surface phenotype with the PR1/HLA-A2 tetramer and anti-CD8. In general, both DC/IGM and T2 cells elicited PR1-CTL. However, T2 were nearly twice as efficient, typically yielding 3% to 6% PR1-CTL (of the bulk culture, determined by tetramer staining) by 4 wk and DC/IGM yielding only 0.5% to 2% PR1-CTL. Since T2 cells may be difficult to use clinically for regulatory reasons, alternative sources of stimulators must be studied.

This procedure is discussed in detail herein and in Molldrem et al. (2003); (2002); (1999); (1998); (1997); each incorporated herein in their entirety by reference). Typical yields of antigen-specific tetramer+ cell numbers were 3% to 5% PR1-CTL using AAPC or T2 cells, but only 0.3% to 2% when DC/IGM are used. These observations are likely to apply to MY4, but all of the comparative experiments will be repeated using this peptide. In addition, serum-free growth conditions will be compared to 10% HS and 5% albumin as a serum substitute in both the DC cultures and the CTL cultures. This will determine the optimal conditions that produce the highest numbers of functional MY4-CTL in the shortest period of time. Weekly tetramer staining during bulk culture restimulations will be used to compare numbers of MY4-CTL, and CFC and cytotoxicity experiments will be used to compare the effector function of the cells.

Additionally, two methods to select antigen-specific CTL from bulk culture will be compared. Although it is predicted that the bulk culture conditions described above will yield CTL that, when adoptively transferred to patients, will not produce significant GVHD, it is possible that GVHD occurs due to the non-specific CTL remaining in the bulk cultures. Since CTL cloning at the end bulk culture is time-consuming and would therefore need to be performed prophylactically in each patient, and because the transfer of CTL clones is not likely to yield long-lived CTL in vivo (Riddell and Greenberg, 1994; Riddell and Greenberg, 1995a; Riddell and Greenberg, 1995b), it is suggested that separation of the peptide-specific fraction from the bulk culture may improve the purity and thereby reduce GVHD. In the first approach to select the antigen-specific CTL, cytokine capture microbead technology developed by Miltenyi Inc., which relies on the ability of antigen-specific CTL to secrete IFN after antigen challenge will be used. These reagents are commercially available, although they are not yet approved for clinical use. MY4-CTL obtained after 4 wk of weekly restimulation will be incubated with MY4 antigen and the bi-specific antibody with anti-CD45 and anti-IFN binding will be co incubated with the cells. A secondary antibody with anti-IFN antibody that is directly linked to microbeads (supplied by Miltenyi, Inc., Germany) will then be used to select out IFN-secreting CTL from bulk culture. In the second approach, the IFN capture method will be compared to a modified peptide/MHC-conjugated bead method developed to select antigen-specific CTL. In previous studies using the PR1 peptide, the feasibility of using PR1 peptide/HLA-A2 monomers linked to streptavidin-coated microbeads (Miltenyi Inc, Germany) to separate antigen-specific CTL from bulk cultures of mixed lymphocytes was demonstrated as described herein (see also Molldrem et al. (2003); (2002); (1999); (1998); (1997), each incorporated by reference herein in their entirety). These studies will be repeated using MY4/HLA-A2 monomers to determine whether similar yields of antigen-specific CTL can be obtained. This methodology will be compared to the standard IFN-capture methodology developed by Miltenyi. Although it is reasonable to expect that the commercial technology will produce sufficient purity of CTL, the device may not capture all CTL with the potential to recognize the cognate peptide/MHC ligand. Since it is unclear whether non-secreting antigen-specific CTL might be required to maintain the more functional fraction in vivo, or whether the non-secreting CTL later become able to express effector function, too few CTL may be selected using the commercial product. Monomer coated beads are likely to capture all of the available antigen-specific CTL from the bulk culture, as shown in previous studies.

### Example 5

### Cyclin E1

To search for other potential tumor-associated antigens, the cyclin E family of proteins were investigated, because it is well known that cyclin E is constitutively expressed in some tumor cells in dependent of the cell cycle, and aberrantly expressing cyclin E contributes to tumorigenesis as a result of chromosomal instability. Cyclin E2 is a homologue of cyclin E1 and both proteins have restricted tissue distribution. To investigate whether cyclin E1 and E2 are over-expressed in hematological malignancy, cyclin E1 and cyclin E2 mRNA expression were first analyzed in 21 patients with hematological malignancy (11 CML (CP), 5 CML (BC), 2 AML, 2 ALL, 1 NHL) and 12 normal donors by RT-PCR. PBMCs from 15 patients and 5 normal donors expressed cyclin E1 mRNA. The relative expression level of cyclin E1 mRNA standardized to β-actin was higher in patients than in normal donors (p=0.0149). Cyclin E2 mRNA was highly over-expressed in PBMCs of patients (10/21) as compared to normal donor PBMCs (0/12; p=0.0109). Next cyclin E1 protein expression was analyzed in 18 of 21 patients with hematological malignancy and 3 of 12 normal donors by western blotting. Although none of PBMCs from normal donor expressed cyclin E1 protein, except one CML (CP) patient, almost all of PBMCs from patients expressed cyclin E1 protein even if they did not express mRNA at the same time point.

Nonameric peptides derived from cyclin E1 and cyclin E2 and predicted to bind to the HLA-A2 allele have similar amino acid sequences, differing only at position 7. Thus, the binding of CCNE1₁₄₄₋₁₅₂ (cyclin E1 derived) and CCNE2v (cyclin E2 derived) was compared to that of PR1 by a peptide-binding assay. The ability of CCNE1₁₄₄₋₁₅₂ and CCNE2₁₄₄₋₁₅₂ to stabilize HLA-A2 on the surface of T2 cells was almost the same as that of PR1 (FI CCNE1₁₄₄₋₁₅₂/FI PR1=1.008, FI CCNE2₁₄₄₋₁₅₂/FI PR1=1.189). To determine whether CCNE1₁₄₄₋₁₅₂ and CCNE2₁₄₄₋₁₅₂ specific CTLs could be elicited in vitro, PBMCs from 7 HLA-A2 positive normal donors were stimulated with peptide-pulsed T2 cells. In 3 of 7 donors, CCNE1₁₄₄₋₁₅₂-stimulated CTL lines killed both CCNE1₁₄₄₋₁₅₂ and CCNE2₁₄₄₋₁₅₂-pulsed T2 cells but not non-peptide-pulsed T2 cells and irrelevant peptide-pulsed T2 cells. In 4 of 7 same donors, CCNE2₁₄₄₋₁₅₂-stimulated CTL lines killed both CCNE2₁₄₄₋₁₅₂ and CCNE1₁₄₄₋₁₅₂-pulsed T2 cells but not non-peptide-pulsed T2 cells and irrelevant peptide-pulsed T2 cells.

Thus, each peptide-specific CTLs can recognize both peptides with HLA-A2, but the immunogenicity of each peptide is different between individuals. Moreover, one of 4 CCNE1₁₄₄₋₁₅₂-stimulated CTL lines which killed CCNE1₁₄₄₋₁₅₂-pulsed T2 cell killed HLA-A2 transfected K562 leukemic cell line which is over-expressing cyclin E1 protein, but not non-transfected K562 leukemic cell line. Thus, CCNE1₁₄₄₋₁₅₂-specific CTL can distinguish leukemic cell lines from normal PBMCs. From the data, it was concluded that cyclin E1/E2 derived peptides are tumor antigens, because 1) cyclin E1/E2 are highly over-expressed in hematological malignancy, 2) cyclin E1/E2 peptides can sufficiently bind to HLA-A2 to stimulate CTL and 3) CCLE1₁₄₄₋₁₅₂ specific CTL preferentially kills leukemic cell lines HLA-A2 restrictively.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### XV. REFERENCES

The following references, to the extent that they provide exemplary procedural or other details supplementary to those set forth herein, are specifically incorporated herein by reference.
U.S. Pat. No. 4,196,265
U.S. Pat. No. 4,435,386
U.S. Pat. No. 4,436,727
U.S. Pat. No. 4,436,728
U.S. Pat. No. 4,505,899
U.S. Pat. No. 4,505,900
U.S. Pat. No. 4,520,019
U.S. Pat. No. 4,554,101
U.S. Pat. No. 4,579,945
U.S. Pat. No. 4,684,611
U.S. Pat. No. 4,797,368
U.S. Pat. No. 4,866,034
U.S. Pat. No. 4,877,611
U.S. Pat. No. 4,879,236
U.S. Pat. No. 4,950,645
U.S. Pat. No. 4,952,500
U.S. Pat. No. 5,139,941
U.S. Pat. No. 5,302,523
U.S. Pat. No. 5,322,783
U.S. Pat. No. 5,384,253
U.S. Pat. No. 5,399,363
U.S. Pat. No. 5,464,765
U.S. Pat. No. 5,466,468
U.S. Pat. No. 5,538,877
U.S. Pat. No. 5,538,880
U.S. Pat. No. 5,543,158
U.S. Pat. No. 5,550,318
U.S. Pat. No. 5,563,055
U.S. Pat. No. 5,580,859
U.S. Pat. No. 5,589,466
U.S. Pat. No. 5,609,870
U.S. Pat. No. 5,610,042
U.S. Pat. No. 5,641,515
U.S. Pat. No. 5,656,610
U.S. Pat. No. 5,693,762
U.S. Pat. No. 5,702,932
U.S. Pat. No. 5,736,524
U.S. Pat. No. 5,739,169
U.S. Pat. No. 5,780,448
U.S. Pat. No. 5,785,970
U.S. Pat. No. 5,789,215
U.S. Pat. No. 5,801,005
U.S. Pat. No. 5,824,311
U.S. Pat. No. 5,830,880
U.S. Pat. No. 5,846,225
U.S. Pat. No. 5,846,233
U.S. Pat. No. 5,846,945
U.S. Pat. No. 5,861,155
U.S. Pat. No. 5,871,986
U.S. Pat. No. 5,945,100
U.S. Pat. No. 5,980,912
U.S. Pat. No. 5,981,274
U.S. Pat. No. 5,994,136
U.S. Pat. No. 5,994,624
U.S. Pat. No. 6,013,516
U.S. Pat. No. 6,020,192
U.S. Pat. No. 6,027,727
U.S. Pat. No. 6,054,297
U.S. Patent Publication No. US 2006/0045883
Akizuki et al., Neoplasia, 9:260-264, 2007.
Alexander-Miller et al., J. Exp. Med., 184:485-492, 1996.
Alexander-Miller et al., J. Exp. Med., 188:1391-1399, 1998.
Alexander-Miller et al., Proc. Natl. Acad. Sci. USA, 93:4102-4107, 1996.
Arap et al., Cancer Res., 55(6):1351-1354, 1995.
Austin-Ward and Villaseca, Rev. Med. Chil., 126(7):838-45, 1998.
Azuma et al., Cell Immunol., 116(1):123-134, 1988.
Baichwal and Sugden, In: Gene Transfer, Kucherlapati (Ed.), NY, Plenum Press, 117-148, 1986.
Bajorin et al., J. Clin. Oncol., 6(5):786-92, 1988.
Bakhshi et al., Cell, 41(3):899-906, 1985.
Benjamini et al., Adv. Exp. Med. Biol., 303:71-77, 1991.
Blomer et al., J. Virol., 71(9):6641-6649, 1997.
Bocchia et al., Blood, 85:2680-2684, 1995.
Bocchia et al., Blood, 87:3587-3592, 1996.
Boon et al., Immunol Today, 18:267-268, 1997.
Boon et al., Important Adv. Oncol., 53-69, 1994.
Braunschweig et al., Blood, 96:3291, 2000.
Brouwer et al., Clin. Exp. Immunol., 98:448-453, 1994.
Brutlag et al., Comput. Appl. Biosci., 6(3):237-245, 1990.
Bukowski et al., Clin. Cancer Res., 4(10):2337-47, 1998.
Burchert et al., Blood, 02:659, 2002.
Caldas et al., Nat. Genet., 8(1):27-32, 1994.
Capaldi et al., Biochem. Biophys. Res. Comm., 74(2):425-433, 1977.
Chen and Okayama, Mol. Cell. Biol., 7(8):2745-2752, 1987.
Chen et al., Biochem. Biophys. Res. Commun., 200:1130-1135, 1994.
Cheng et al., Cancer Res., 54(21):5547-5551, 1994a.
Chou and Fasman, Adv. Enzymol., 47:45-148, 1978b.
Chou and Fasman, Annu. Rev. Biochem., 47:251-276, 1978a.
Chou and Fasman, Biochemistry, 13(2):211-222, 1974b.
Chou and Fasman, Biophys. J., 26(3):385-399, 1979.
Christodoulides et al., Microbiology, 144(Pt 11):3027-37, 1998.
Cleary and Sklar, Proc. Natl. Acad. Sci. USA, (21):7439-7443, 1985.
Cleary et al., J. Exp. Med., 164(1):315-320, 1986.
Cotten et al., Proc. Natl. Acad. Sci. USA, 89(13):6094-6098, 1992.
Coupar et al., Gene, 68:1-10, 1988.
Culver et al., Science, 256(5063):1550-1552, 1992.
Curiel, Nat. Immun., 13(2-3):141-164, 1994.
Davidson et al., J. Immunother., 21(5):389-98, 1998.
den Haan et al., Science, 268:1476-1480, 1995.
den Haan et al., Science, 279:1054-1057, 1998.
Dermime et al., Blood, 86, 1995.
Dermime et al., Bone Marrow Transplant., 19:989-999, 1997.
Desmedt et al., Int J Cancer, 119:2539-2545, 2006.
Dillman, Cancer Biother. Radiopharm., 14(1):5-10, 1999.
Fechheimer et al., Proc. Natl. Acad. Sci. USA, 84:8463-8467, 1987.
Fetrow and Bryant, Biotechnology, 11(4):479-484, 1993.
Finlay et al., Endocrinology, 133:996-1002, 1993.
Foekens J A, Ries C, Look M P, Gippner-Steppert C, Klijn J G, Jochum M. The prognostic value of polymorphonuclear leukocyte elastase in patients with primary breast cancer. Cancer Res. 2003; 63:337-341.
Fraley et al., Proc. Natl. Acad. Sci. USA, 76:3348-3352, 1979.
Franssen et al., Kidney Int., 57:2195-1206, 2000.
Franssen et al., Lancet., 347:116, 1996.
Friedmann, Science, 244:1275-1281, 1989.
Gao et al., Blood, 95:2198-2203, 2000.
Gopal, Mol. Cell. Biol., 5:1188-1190, 1985.
Goulmy et al., N. Engl. J. Med., 334:281-285, 1996.
Graham and Van Der Eb, Virology, 52:456-467, 1973.
Grunhaus and Horwitz, Seminar in Virology, 3:237-252, 1992.
Hanibuchi et al., Int. J. Cancer, 78(4):480-485, 1998.
Harlan and Weintraub, J. Cell Biol., 101:1094-1099, 1985.
Harlow and Lane, Antibodies: A Laboratory Manual. Cold Spring Harbor Laboratory Press, Cold Spring harbor, NY, 553-612, 1988.
Harwell et al., Cancer Res., 60:481-489, 2000.
Hellstrand et al., Acta Oncol., 37(4):347-353, 1998.
Hensel et al., Bone Marrow Transplant., 23:71-78, 1999.
Horwich et al. J. Virol., 64:642-650, 1990.
Hui and Hashimoto, Infect. Immun., 66(11):5329-36, 1998.
Husson et al., J. Bacteriol., 172(2):519-524, 1990.
Hussussian et al., Nat. Genet., 8(1):15-21, 1994.
Irie and Morton, Proc. Natl. Acad. Sci. USA, 83(22):8694-8698, 1986.
Irie et al., Lancet., 1(8641):786-787, 1989.
Jacobs et al., Nature, 327(6122):532-535, 1987.
Jameson and Wolf, Comput. Appl. Biosci., 4(1):181-186, 1988.
Janetzki et al., J. Immunol. Methods, 234:1-12, 2000.
Johnson et al., In: Biotechnology And Pharmacy, Pezzuto et al. (Eds.), Chapman and Hall, NY, 1993.
Ju et al., J. Neuropathol. Exp. Neurol., 59(3):241-50, 2000.
Kaeppler et al., Plant Cell Reports, 9:415-418, 1990.
Kamb et al., Nat. Genet., 8(1):23-2, 1994.
Kamb et al., Science, 2674:436-440, 1994.
Kaneda et al., Science, 243:375-378, 1989.
Kato et al, J. Biol. Chem., 266:3361-3364, 1991.
Kelleher and Vos, Biotechniques, 17(6):1110-7, 1994.
Kerr et al., Br. J. Cancer, 26(4):239-257, 1972.
Kochenderfer and Molldrem, Curr. Oncol. Rep. 3:193-200, 2001.
Kochenderfer et al., Blood, 100:3639-3645, 2002.
Kyte and Doolittle, J. Mol. Biol., 57(1):105-32, 1982.
Laughlin et al., J. Virol., 60(2):515-524, 1986.
Lebkowski et al., Mol. Cell. Biol., 8(10):3988-3996, 1988.
Lotte et al., Adv. Tuberc. Res., 21:107-93; 194-245, 1984.
Luelmo, Am. Rev. Respir. Dis., 125(3 Pt 2):70-72, 1982.
Mann et al., Cell, 33:153-159, 1983.
Marijt et al., Blood 82:3778-3785, 1995.
Martin et al., Nature, 345(6277):739-743, 1990.
McLaughlin et al., J. Virol., 62(6):1963-1973, 1988.
Miller et al., Am. J. Clin. Oncol., 15(3):216-221, 1992.
Mitchell et al., Ann. NY Acad. Sci., 690:153-166, 1993.
Mitchell et al., J. Clin. Oncol., 8(5):856-869, 1990.
Molldrem et al., Blood, 88:2450-2457, 1996.
Molldrem et al., Blood, 90:1858, 1998.
Molldrem et al., Blood, 90:2529-2534, 1997.
Molldrem et al., Br. J. Haematol., 102:1314-1322, 1998.
Molldrem et al., Cancer Res., 59:2675-2681, 1999.
Molldrem et al., Curr. Opin. Hematol., 9:503-508, 2002.
Molldrem et al., J. Clin. Invest., 111:639-647, 2003.
Molldrem et al., Nat. Med., 6:1018-1023, 2000.
Molldrem et al., Oncogene, 21:8668-8673, 2002.
Mori et al., Cancer Res., 54(13):3396-3397, 1994.
Morton et al., Arch. Surg., 127:392-399, 1992.
Muzyczka, Curr. Topics Microbiol. Immunol., 158:97-129, 1992.
Naldini et al., Science, 272(5259):263-267, 1996.
Nanda and Sercarz, Cell, 82:13-17, 1995.
Nestle et al., Nature Med., 4:328-332, 1998.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Okamoto et al., Proc. Natl. Acad. Sci. USA, 1(23):11045-11049, 1994.
Omirulleh et al., Plant Mol. Biol., 21(3):415-28, 1993.
Orlow et al., Cancer Res, 54(11):2848-2851, 1994.
Pardoll, Current Opin. Immunol., 14:619-623, 1992.
Pardoll, Nature Rev. Immunol., 2:227-238, 2002.
Pardoll, Nature, 369:357, 1994.
Parker et al., J. Immunol., 152:163, 1994.
Paskind et al., Virology, 67:242-248, 1975.
PCT Appln, WO 91/16347
PCT Appln. WO 94/09699
PCT Appln. WO 95/06128
Pietras et al., Oncogene, 17(17):2235-49, 1998.
Pinilla-Ibarz et al., Blood, 95:1781-1787, 2000.
Porter et al., Mol Cell Biol., 21:6254-6269, 2001.
Potrykus et al., Mol. Gen. Genet., 199:183-188, 1985.
Qin et al., Proc. Natl. Acad. Sci. USA, 95(24):14411-14416, 1998.
Rabinovich et al., Science, 265(5177):1401-1404, 1994.
Ravindranath and Morton, Intern. Rev. Immunol., 7: 303-329, 1991.
Remington's Pharmaceutical Sciences, 15^{th} ed., pages 1035-1038 and 1570-1580, Mack Publishing Company, Easton, Pa., 1980.
Riddell and Greenberg, Annu. Rev. Immunol., 13:545-586, 1995.
Riddell and Greenberg, Cancer Treat Res., 76:337-369, 1995.
Riddell and Greenberg, Curr. Top Microbiol. Immunol., 189:9-34, 1994.
Riddell et al., Science, 257:238-241, 1992.
Ridgeway, In: Vectors: A survey of molecular cloning vectors and their uses, Rodriguez and Denhardt (Eds.), Stoneham:Butterworth, 467-492, 1988.
Rippe et al., Mol. Cell. Biol., 10:689-695, 1990.
Rosenberg and White, J. Immunother. Emphasis. Tumor Immunol., 19:81-84, 1996.
Rosenberg et al., Ann. Surg. 210(4):474-548, 1989.
Rosenberg et al., Nature Med., 4:321-327, 1998.
Rosenberg et al., N. Engl. J. Med., 319:1676, 1988.
Roux et al., Proc Natl Acad Sci USA. 1989 December; 86(23):9079-83.
Salter and Cresswell, Embo. J., 5:943-949, 1986.
Santini et al., J. Exp. Med., 191:1777-1788, 2000.
Sato et al., Surg Oncol. 15:217-222, 2006.
Savage et al., Immunity, 10:485-492, 1999.
Scheibenbogen et al., Blood, 100:2132-2137, 2002.
Scheibenbogen et al., Int. J. Cancer, 71:932-936, 1997.
Serrano et al., Nature, 366:704-707, 1993.
Serrano et al., Science, 267(5195):249-252, 1995.
Snapper et al., Proc. Natl. Acad. Sci. USA, 85(18):6987-6991, 1988.
Sturrock et al., J. Biol. Chem., 267:21193, 1992.
Takada et al., J. Clin. Microbiol., 33(3):658-660, 1995.
Temin, In: Gene Transfer, Kucherlapati (Ed.), NY, Plenum Press, 149-188, 1986.
Tratschin et al., Mol. Cell. Biol., 4:2072-2081, 1984.
Tsujimoto and Croce, Proc. Natl. Acad. Sci. USA, 83(14):5214-5218, 1986.
Tsujimoto et al., Science, 228(4706):1440-1443, 1985.
Verdijk et al., J. Immunol., 163:57-61, 1999.
Wada et al., Nucleic Acids Res. 18:2367-2411, 1990.
Wang et al., Biol. Blood Marrow Transplant., 6:118, 2000.
Weinberger et al., Science, 228:740-742, 1985.
Williams et al., J. Immunol., 152:4722-4732, 1994.
Wolf et al., Comput. Appl. Biosci., 4(1):187-191, 1988.
Wong et al., Gene, 10:87-94, 1980.
Yamamoto et al., Jpn. J. Cancer Res., 79:866-873, 1988.
Yamashita et al., Br J Cancer, 69:72-76, 1994.
Yee et al., J. Immunol., 162:2227-2234, 1999.
Yin et al., J. Biol. Resp. Modif., 8:190-205, 1989.
Zufferey et al., Nat. Biotechnol., 15(9):871-875, 1997.

1. A method for treating or preventing breast cancer in an individual comprising administering to said individual a therapeutically effective amount of an immunogenic composition comprising a PR1 peptide, a PR1-derived peptide, a PR3 peptide, a myeloperoxidase peptide, a cyclin E1 peptide, a cyclin E2 peptide, or a mixture thereof.

2. The method of claim 1, wherein the method comprises administering the immunogenic composition more than once.

3. The method of claim 1, wherein the therapeutically effective amount is in the range of 0.20 mg to 5.0 mg, 0.025 mg to 1.0 mg, or 2.0 mg to 5.0 mg.

4. (canceled)

5. (canceled)

6. The method of claim 1, wherein the immunogenic composition further comprises an adjuvant.

7. The method of claim 6, wherein the immunogenic composition and adjuvant are injected subcutaneously into the individual.

8. The method of claim 7, wherein an immunomodulatory agent is further injected subcutaneously into the individual.

9. The method of claim 1, wherein the peptide is further defined as a HLA-A2 restricted peptide.

10. (canceled)

11. (canceled)

12. (canceled)

13. (canceled)

14. (canceled)

15. The method of claim 1, wherein the immunogenic composition is administered by an antigen-presenting cell pulsed or loaded with the peptide.

16. The method of claim 15, wherein the antigen presenting cell is a dendritic cell.

17. The method of claim 15, wherein the antigen-presenting cell contains one or more peptide.

18. The method of claim 1, further comprising treating the individual with a second anticancer agent, wherein the second anticancer agent is a therapeutic polypeptide, a nucleic acid encoding a therapeutic polypeptide, a chemotherapeutic agent, a biological and/or small molecule targeted agent, an immunomodulatory agent, or a radiotherapeutic agent.

19. (canceled)

20. (canceled)

21. (canceled)

22. (canceled)

23. (canceled)

24. A method for treating or preventing breast cancer in an individual comprising:
(a) contacting CTLs of said individual with a PR1 peptide, a PR1-derived peptide, a PR3 peptide, a myeloperoxidase peptide, a cyclin E1 peptide, a cyclin E2 peptide, or a mixture thereof; and
(b) administering a therapeutically effective amount of the CTLs of step (a) to the patient.

25. The method of claim 24, further comprising expanding said CTL's by ex vivo or in vivo methods prior to administration.

26. (canceled)

27. (canceled)

28. The method of claim 24, wherein contacting comprises providing an antigen presenting cell loaded with said peptide or that expresses said peptide from an expression construct.

29. The method of claim 24, further comprising providing CTLs transfected with a T cell receptor specific for the peptide.

30. The method of claim 24, wherein the therapeutically effective amount of CTL cells required to provide therapeutic benefit is from about 0.1×10⁵ to about 5×10⁷ cells per kilogram weight of the subject.

31. The method of claim 24, wherein the peptide is further defined as a HLA-A2 restricted peptide.

32. A method for treating or preventing a cancer in an individual comprising administering to said individual a therapeutically effective amount of an immunogenic composition comprising an expression construct encoding PR1 peptide, a PR1-derived peptide, a PR3 peptide, a myeloperoxidase peptide, a cyclin E1 peptide, a cyclin E2 peptide, or a mixture thereof.

33. (canceled)

34. (canceled)

35. The method of claim 32, wherein said expression construct encodes a second tumor associated peptide.

36. (canceled)

37. The method of claim 32, wherein the peptide is further defined as a HLA-A2 restricted peptide.

38. (canceled)

39. A composition comprising:
PR1 peptide, a PR1-derived peptide, a PR3 peptide, a myeloperoxidase peptide, a cyclin E1 peptide, a cyclin E2 peptide, or a mixture thereof; and
a second anti-breast cancer agent.

40. (canceled)

41. (canceled)

42. (canceled)

43. (canceled)

44. (canceled)

45. (canceled)

46. (canceled)

47. (canceled)


SEQUENCE LISTING (35 sequences)

SEQ ID NO: 1 - protein, 9 aa, Artificial Sequence
  Synthetic Peptide
VLQELNVTV

SEQ ID NO: 2 - protein, 8 aa, Artificial Sequence
  Synthetic Peptide
VLQELWTV

SEQ ID NO: 3 - protein, 9 aa, Artificial Sequence
  Synthetic Peptide
VLQELNVKV

SEQ ID NO: 4 - protein, 8 aa, Artificial Sequence
  Synthetic Peptide
VLQELWKV

SEQ ID NO: 5 - protein, 8 aa, Artificial Sequence
  Synthetic Peptide
VMQELWTV

SEQ ID NO: 6 - protein, 9 aa, Artificial Sequence
  Synthetic Peptide
RLFEQVMRI

SEQ ID NO: 7 - protein, 9 aa, Artificial Sequence
  Synthetic Peptide
SLWRRPFNV

SEQ ID NO: 8 - protein, 9 aa, Artificial Sequence
  Synthetic Peptide
KLLLALAGL

SEQ ID NO: 9 - protein, 9 aa, Artificial Sequence
  Synthetic Peptide
LLSYFKQPV

SEQ ID NO: 10 - protein, 9 aa, Artificial Sequence
  Synthetic Peptide
LIQPFMFRL

SEQ ID NO: 11 - protein, 9 aa, Artificial Sequence
  Synthetic Peptide
RVFFASWRV

SEQ ID NO: 12 - protein, 9 aa, Artificial Sequence
  Synthetic Peptide
VLGEVDTSL

SEQ ID NO: 13 - protein, 9 aa, Artificial Sequence
  Synthetic Peptide
LLLREHNRL

SEQ ID NO: 14 - protein, 9 aa, Artificial Sequence
  Synthetic Peptide
VLTPAQLNV

SEQ ID NO: 15 - protein, 9 aa, Artificial Sequence
  Synthetic Peptide
YLHVALDLL

SEQ ID NO: 16 - protein, 9 aa, Artificial Sequence
  Synthetic Peptide
VLGPTAMRK

SEQ ID NO: 17 - protein, 9 aa, Artificial Sequence
  Synthetic Peptide
GLPGYNAWR

SEQ ID NO: 18 - protein, 9 aa, Artificial Sequence
  Synthetic Peptide
TLIQPFMFR

SEQ ID NO: 19 - protein, 9 aa, Artificial Sequence
  Synthetic Peptide
RLFEQVMRI

SEQ ID NO: 20 - protein, 9 aa, Artificial Sequence
  Synthetic Peptide
VLSSMEEAK

SEQ ID NO: 21 - protein, 9 aa, Artificial Sequence
  Synthetic Peptide
FMFRLDNRY

SEQ ID NO: 22 - protein, 9 aa, Artificial Sequence
  Synthetic Peptide
AMRKYLPTY

SEQ ID NO: 23 - protein, 9 aa, Artificial Sequence
  Synthetic Peptide
AMVQIITYR

SEQ ID NO: 24 - protein, 9 aa, Artificial Sequence
  Synthetic Peptide
CIIGTQFRK

SEQ ID NO: 25 - protein, 9 aa, Artificial Sequence
  Synthetic Peptide
GLLAVNQRF

SEQ ID NO: 26 - protein, 9 aa, Artificial Sequence
  Synthetic Peptide
NPRWDGERL

SEQ ID NO: 27 - protein, 9 aa, Artificial Sequence
  Synthetic Peptide
IVRFPTDQL

SEQ ID NO: 28 - protein, 9 aa, Artificial Sequence
  Synthetic Peptide
GPTAMRKYL

SEQ ID NO: 29 - protein, 9 aa, Artificial Sequence
  Synthetic Peptide
RPFNVTDVL

SEQ ID NO: 30 - protein, 9 aa, Artificial Sequence
  Synthetic Peptide
QVMRIGLDL

SEQ ID NO: 31 - protein, 9 aa, Artificial Sequence
  Synthetic Peptide
NPRVPLSRV

SEQ ID NO: 32 - protein, 9 aa, Artificial Sequence
  Synthetic Peptide
MQRSRDHGL

SEQ ID NO: 33 - protein, 9 aa, Artificial Sequence
  Synthetic Peptide
SNRAFVRWL

SEQ ID NO: 34 - protein, 9 aa, Artificial Sequence
  Synthetic Peptide
TIRNQINAL

SEQ ID NO: 35 - protein, 9 aa, Artificial Sequence
  Synthetic Peptide
NLRNMSNQL